(19) United States
(12) Patent Application Publication
(10) Pub. No.: US 2026/0297608 A1
(43) Pub. Date: Oct. 1, 2026

(54) METHOD FOR CULTIVATING HUANGLONGBING-RESISTANT PLANTS AND APPLICATION THEREOF

(71) Applicant: INSTITUTE OF MICROBIOLOGY, CHINESE ACADEMY OF SCIENCES, Beijing (CN)

(72) Inventors: Jian Ye, Beijing (CN); Pingzhi Zhao, Beijing (CN); Huan Yang, Beijing (CN)

(73) Assignee: INSTITUTE OF MICROBIOLOGY, CHINESE ACADEMY OF SCIENCES, Beijing (CN)

(21) Appl. No.: 19/383,337

(22) Filed: Nov. 7, 2025

Related U.S. Application Data
(63) Continuation of application No. PCT/CN2025/084478, filed on Mar. 24, 2025.

(30) Foreign Application Priority Data
Jan. 26, 2025 (CN) 202510122567.4

(51) Int. Cl. C12N 15/82; C12N 9/22; C12N 15/11

(52) U.S. Cl. CPC C12N 15/8281; C12N 9/226; C12N 15/111; C12N 15/8213; C12N 2310/20

(57) ABSTRACT

The present disclosure discloses a citrus genus gene fragment related to Huanglongbing resistance of plants and application thereof. The gene promoter fragment has a nucleotide sequence as shown in SEQ ID NO. 1, and includes a plurality of characteristic motifs that bind to a disease-resistant transcription factor MYC2. The transcription factor MYC2 can bind to the characteristic motifs and exert transcription factor activities to transcriptively activate expression of a plant Huanglongbing susceptibility gene PUB21. The present disclosure clarifies a tight gene feedback loop formed by the susceptibility gene PUB21 and a ubiquitinated substrate thereof, i.e. the transcription factor MYC2, and provides a breeding strategy for enhancing disease resistance of plants by reducing and/or eliminating an expression level of the susceptibility gene PUB21. The resistance breeding strategy can effectively resist replication and spread of a Huanglongbing pathogen and is of great significance to prevention and control of Huanglongbing in plants.

### CROSS-REFERENCE TO RELATED APPLICATION

The present application claims priority to the Chinese Patent Application No. CN202510122567.4 filed on Jan. 26, 2025, the entire contents of which are incorporated herein by reference.

### SEQUENCE LISTING

This application contains a sequence listing in computer readable form (File name: 19234018001.xml; date of creation: Nov. 7, 2025; File size: 26,524 bytes) which is incorporated herein by reference in its entirety and forms part of the disclosure.

### TECHNICAL FIELD

The present disclosure belongs to the field of biotechnology, and specifically relates to a citrus or Nicotiana genus gene fragment associated with Huanglongbing resistance of plants, and application thereof, in particular to identification and functional verification of fragments within a PUB21 promoter region of a Huanglongbing susceptibility gene of plants, and application thereof in regulating Huanglongbing resistance of the plants.

### BACKGROUND

HLB is primarily caused by a phloem-limited bacterium, Candidatus Liberibacter asiaticus (CLas), which has an extremely broad host range, is mainly transmitted by an insect vector, Asian Diaphorina citri Kuwayama and may also survive in fruit fly and Aedes mosquito cells. The HLB pathogen CLas infects citrus genus plants and related genera of the Aurantioideae subfamily of the Rutaceae family. All commercial citrus cultivars are susceptible to HLB. Citrus Huanglongbing, also known as citrus greening disease, is currently the most destructive citrus disease, causing severe economic losses to the global citrus industry. Beyond citrus genus plants, the HLB pathogen has been detected in related genera including trifoliate oranges, Kumquat, Clausena lansium, Murraya paniculata, Murraya paniculata, Wood Apple, and Atalantia buxifolia. Beyond plants of the Rutaceae family, the HLB bacteria can also be transmitted via dodder to non-Rutaceae plants such as Vinca, tobacco, and tomatoes.

As sessile organisms, plants face multiple biotic and abiotic stresses throughout the life cycles. Through long-term evolution, plants have developed complex defense mechanisms to combat pathogen infection. Protein degradation plays a crucial role in plant defense, with a ubiquitin-proteasome system serving as one of primary mechanisms. Within the system, E3 ligases are key components that determine the specificity of a substrate and mediate ubiquitination degradation of the substrate. A plant U-box protein (PUB) family, as one such component, has been reported in recent years to play a crucial role in interactions between various plants including rice and Arabidopsis and pathogens. Multiple pathogens manipulate PUBs to target and degrade disease-resistant proteins, thereby undermining host immunity. Several genes encoding plant U-box (PUB)-type E3 exhibit higher expression levels in disease-susceptible C. sinensis compared to disease-tolerant C. daoxianensis and Microcitrusaustralasica. Among these, PUB21 shows the highest upregulation during CLas infection, while no significant transcriptional response was observed in M. australasica that is more tolerant to HLB. Therefore, strict regulation of PUBs protein expression levels plays a crucial role in resistance of plants to pests and diseases.

As fundamental elements of gene expression, promoters play a key role in regulating gene expression levels. Studying regulation patterns of promoters or comparing differences of promoter characteristics to precisely regulate expression levels of target genes is important for guiding next-generation resistance breeding. In promoters, insertion-deletion polymorphisms may be caused by transposon insertion, which can induce PUB protein expression and precisely regulate the expression levels of target genes. This process is crucial for guiding next-generation resistance breeding.

Transposons, also known as transposable elements, are DNA sequences that can move independently on host genomes, typically ranging from 100 bp to 10,000 bp in length, which is similar to viruses. At present, transposons are primarily divided into three classes based on replication types thereof: Class I transposons, also known as retrotransposons, are of a “copy-paste” type; Class II transposons, shortly referred to as transposons, are of a “cut-paste” type; and Helitron transposons, which perform transposition through a “rolling ring” replication method, are a novel transposon discovered in the last 20 years. Autonomous Helitron can encode replicase Rep and helicase Hel, and expand in a genome through a rolling ring replication method similar to DNA viruses. Due to the specific replication characteristics, the Helitron transposons have become a hot topic in contemporary biological research. For example, (I) a gene transcription regulation network is altered based on Helitron to integrate and amplify a transcription factor expression regulation region, so as to regulate the expression of nearby genes; and (II) gene epigenetic modifications are altered based on Helitron, and an siRNA produced by Helitron may target donor genes to cause gene epigenetic modifications to inhibit gene expression.

To date, no effective cure method or resistance gene for Huanglongbing has been discovered, leaving the citrus industry globally vulnerable to severe economic losses caused by Huanglongbing. How to further complete identification and functional verification of transposon fragments within a promoter region of HLB susceptibility genes on the basis of determination of the HLB susceptibility genes, especially to verify application thereof in regulating the Huanglongbing resistance of citrus, is a highly valuable topic.

### SUMMARY OF THE INVENTION

The technical problem to be solved by the present disclosure is how to enhance Huanglongbing resistance of plants so as to cultivate Huanglongbing-resistant plants, plant parts or plant cells. The technical problems to be solved are not limited to the described technical subjects, and those skilled in the art can clearly understand other technical subjects not mentioned herein through the following description.

To solve the aforementioned technical problems, the present disclosure first provides a DNA molecule.

The DNA molecule provided by the present disclosure is a fragment located within a PUB21 promoter region of a plant Huanglongbing susceptibility gene, and is any one of a1)-a3) as follows:
a1) a DNA molecule as shown in SEQ ID No. 1;
a2) a DNA molecule having a same function, obtained by reducing or eliminating an expression and/or function of a full-length or partial fragment of the DNA molecule defined in a1); and
a3) a DNA molecule that is at least 70%, at least 75%, at least 80%, at least 85%, at least 90%, at least 95%, at least 96%, at least 97%, at least 98% or at least 99% identical to and has a same function as the DNA molecule defined in a1).

In a2), the full-length or partial fragment of the DNA molecule includes a characteristic motif that binds to a transcription factor MYC2.

In a2), the reduction or elimination of the expression and/or function of the full-length or partial fragment of the DNA molecule refers to treating the full-length or partial fragment of the DNA molecule with gene knockout technology, gene silencing technology, inactivation mutation technology, PROTAC technology or small molecule inhibitors.

In a2), the reduction or elimination of the expression and/or function of the full-length or partial fragment of the DNA molecule refers to replacement and/or insertion and/or deletion of one or more bases of the DNA molecule. The replacement and/or insertion and/or deletion of the one or several bases refer(s) to replacement and/or insertion and/or deletion of no more than 10 bases.

In a3), identity refers to sequence similarity with a natural nucleic acid sequence. Identity may be evaluated by the naked eye or computer software. When the computer software is used, the identity between two or more sequences may be expressed as a percentage (%) which may be used to evaluate the identity between related sequences.

One of ordinary skill in the art can easily mutate a nucleotide sequence of the DNA molecule of the present disclosure using known methods, such as directed evolution and point mutation. Those nucleotides which have been artificially modified to have 70% or higher identity to the nucleotide sequence of the DNA molecule provided by the present disclosure, provided that they have the same function, are nucleotide sequences derived from the present disclosure and are equivalent to the sequences of the present disclosure.

The DNA molecule described above includes a plurality of characteristic motifs (G-box like: CANNTG) that bind to the transcription factor MYC2. The transcription factor MYC2 can bind to the characteristic motifs and exert the transcription factor activity to transcriptively activate the expression of PUB21, forming a tight feedback loop ligating the PUB21 promoter-PUB21-MYC2, and participating in the regulation of Huanglongbing resistance.

The present disclosure further provides a method for preparing the DNA molecule described above. The method includes treating characteristic motifs in full-length or partial fragments of most DNA molecules that bind to a transcription factor MYC2 with gene knockout technology, gene silencing technology, inactivation mutation technology, PROTAC technology or small molecule inhibitors.

In the method for preparing the DNA molecule described above, the gene knockout technology includes zinc finger nucleases (ZFN) technology, transcription activator-like effector (TALE) technology, CRISPR/Cas technology or TALE-CRISPR/Cas technology.

The CRISPR/Cas technology is selected from CRISPR/Cas3, CRISPR/Cas9, CRISPR/Cas12, CRISPR/Cas12a, CRISPR/Cas12b, CRISPR/Cas13, CRISPR/Cas13a, CRISPR/Cas13b, CRISPR/Cas13c, CRISPR/Cas13e, CRISPR/Cas13f, CRISPR/CasX or CRISPR/IscB systems.

In one implementation of the present disclosure, the gene knockout technology employs a CRISPR/Cas9 gene editing vector. The CRISPR/Cas9 gene editing vector expresses an sgRNA and Cas9 protein targeting the full-length or partial fragment of the DNA molecule described above.

In one implementation of the present disclosure, the CRISPR/Cas9 gene editing vector includes an sgRNA gene expression cassette and a Cas9 gene expression cassette.

Further, the sgRNA gene expression cassette includes a promoter for initiating expression of an sgRNA gene. The promoter for initiating the expression of the sgRNA gene is preferably a CsU6 promoter.

Further, the Cas9 gene expression cassette includes a promoter for initiating expression of a Cas9 gene. The promoter for initiating the expression of the Cas9 gene is preferably a CmYLCV promoter.

Furthermore, a target sequence of the sgRNA is as shown in positions 1105-1124 of the sequence SEQ ID No: 1.

A nucleotide sequence of the CsU6 promoter is as shown in a sequence SEQ ID No. 2.

A nucleotide sequence of the CmYLCV promoter is as shown in a sequence SEQ ID No. 3.

In one implementation of the present disclosure, the method for preparing the DNA molecule described above includes mutating the characteristic motif in the full-length or partial fragment of the DNA molecule described above that binds to the transcription factor MYC2, so as to prevent the transcription factor MYC2 from binding to the characteristic motif.

In one implementation of the present disclosure, a mutation form may be deletion mutation and/or insertion mutation and/or base substitution.

In order to solve the technical problems described above, the present disclosure further provides a gene regulation system. The gene regulation system is configured to prepare the DNA molecule described above.

The gene regulation system treats characteristic motifs in full-length or partial fragments of most DNA molecules that bind to a transcription factor MYC2 with gene knockout technology, gene silencing technology, inactivation mutation technology, PROTAC technology or small molecule inhibitors.

The gene regulation system includes a nucleic acid molecule and an enzyme protein, wherein the nucleic acid molecule is an sgRNA molecule, and the enzyme protein is a Cas protein or an ortholog of Cas. The enzyme protein may be selected from Cas3, Cas9, Cas12a, Cas12b, Cas13a, Cas13b, Cas13c, Cas13e, Cas13f, CasX, or IscB proteins or orthologs thereof.

In one implementation of the present disclosure, a vector used in the gene regulation system is a CRISPR/Cas9 gene editing vector that expresses an sgRNA and Cas9 protein targeting the full-length or partial fragment of the DNA molecule described above.

Furthermore, the CRISPR/Cas9 gene editing vector includes an sgRNA gene expression cassette and a Cas9 gene expression cassette.

The sgRNA gene expression cassette includes a promoter for initiating expression of an sgRNA gene. The promoter for initiating the expression of the sgRNA gene is preferably a CsU6 promoter.

The Cas9 gene expression cassette includes a promoter for initiating expression of a Cas9 gene. The promoter for initiating the expression of the Cas9 gene is preferably a CmYLCV promoter.

Furthermore, a target sequence of the sgRNA is as shown in positions 1105-1124 of a sequence SEQ ID No. 1.

A nucleotide sequence of the CsU6 promoter is as shown in a sequence SEQ ID No. 2.

A nucleotide sequence of the CmYLCV promoter is as shown in a sequence SEQ ID No. 3.

In order to solve the technical problems described above, the present disclosure further provides a kit including the gene regulation system described above.

The present disclosure further provides a PUB21 promoter. The PUB21 promoter includes the DNA molecule described above.

In one implementation of the present disclosure, the PUB21 promoter may be of a Helitron type, such as CsPUB21.

In another implementation of the present disclosure, the PUB21 promoter is of a non-Helitron type, including CiPUB21, MpPUB21 and BkPUB21, etc.

In order to solve the above technical problems, the present disclosure further provides new use of a substance that reduces or eliminates an expression and/or function of a full-length or partial fragment of the DNA molecule described above, or the gene regulation system described above or the kit including the gene regulation system. The use includes any one of A1)-A6) as follows:
A1) enhancing Huanglongbing resistance of plants, plant parts, or plant cells;
A2) preparation of products for enhancing Huanglongbing resistance of plants, plant parts, or plant cells;
A3) cultivation of Huanglongbing-resistant plants, plant parts, or plant cells;
A4) preparation of products for cultivating Huanglongbing-resistant plants, plant parts, or plant cells;
A5) prevention and control of Huanglongbing in plants, plant parts, or plant cells; and
A6) preparation of products for preventing and controlling Huanglongbing in plants, plant parts, or plant cells.

In order to solve the technical problems described above, the present disclosure further provides a method for enhancing Huanglongbing resistance of plants, plant parts or plant cells or for cultivating Huanglongbing-resistant plants, plant parts or plant cells.

The method for enhancing Huanglongbing resistance of plants, plant parts, or plant cells or for cultivating Huanglongbing-resistant plants, plant parts, or plant cells according to the present disclosure includes administering to recipient plants, plant parts, or plant cells the gene regulation system or the kit, such that an expression and/or function of a full-length or partial fragment of the DNA molecule in a PUB21 gene promoter in the recipient plants, plant parts, or plant cells are/is reduced or eliminated.

Further, the method for reducing or eliminating the expression and/or function of the full-length or partial fragment of the DNA molecule described above in the recipient plants, plant parts or plant cells is to introduce substances that reduce or eliminate the expression and/or function of the full-length or partial fragment of the DNA molecule described above into the recipient plants.

The method for enhancing Huanglongbing resistance of plants, plant parts, or plant cells or for cultivating Huanglongbing-resistant plants, plant parts, or plant cells according to the present disclosure includes the step of deleting positions 1113-1196 of the PUB21 gene promoter in genome DNA of the recipient plants.

In some implementations of the present disclosure, the deletion is homozygous substitution, that is, the same deletion occurs in homologous chromosomes.

A sequence of any one of the characteristic motifs (G-box like) that bind to the transcription factor MYC2 described above is as follows: CANNTG.

Any one of the PUB21 gene promoters described above is a PUB21 gene promoter of the Citrus or Nicotiana genus. Sequences of the PUB21 gene promoter are as shown in sequences SEQ ID No. 1, SEQ ID No. 8 and SEQ ID No. 9.

Any one of the transcription factors MYC2 described above is a transcription factor CsMYC2. An amino acid sequence and coding sequence (CDS) of the transcription factor CsMYC2 are recorded in the invention patent application documents with the publication number CN115160421A.

In any one of the applications or methods described above, the plants are monocotyledonous or dicotyledonous plants.

The dicotyledonous plants are plants of the order Rutales or Solanales.

The plants of the order Rutales are plants of the Rutaceae family.

The plants of the order Solanales are plants of the Solanaceae family.

The plants of the Rutaceae family are Citrus genus plants.

The plants of the Solanaceae family are Nicotiana genus plants.

The Citrus genus plants are citrus.

The Nicotiana genus plants are tobacco.

In some implementations of the present disclosure, the citrus may be at least one of the following varieties: C. sinensis, C. reticulata, C. daoxianensis, C. linwuensis, C. mangshanensis, C. ichangensis, C. hongheensis, C. maxima, C. medica, P. trifoliata, or M. australasica.

According to the present disclosure, a multi-sequence alignment method is used, and by conducting genomic sequence alignment analysis on Huanglongbing-susceptible species (C. sinensis and C. reticulata), Huanglongbing-tolerant species (C. daoxianensis, C. linwuensis, C. mangshanensis, P. trifoliata, C. ichangensis, C. hongheensis, C. maxima, C. medica and M. australasica) and Huanglongbing-resistant species (Murraya paniculata and Murraya koenigii), it is identified that 150-bp Helitron transposon insertion exists within a susceptible gene PUB21 promoter region in the Huanglongbing-susceptible species and the Huanglongbing-tolerant species. Meanwhile, the present disclosure further identifies that the citrus genus PUB21 promoter region and a Helitron transposon fragment thereof include a plurality of DNA motifs (G-box like: CANNTG) that bind to the transcription factor MYC2. The transcription factor MYC2 can bind to the motifs and exert transcription factor activities to transcriptionally activate the expression of the PUB21 gene, thereby forming a tight feedback loop: PUB21pro-PUB21-MYC2. The present disclosure for the first time identifies the presence of DNA transposon Helitron insertion within a gene PUB21 promoter (PUB21pro) of citrus E3 ubiquitin ligase, clarifies the tight feedback loop formed by PUB21 and a ubiquitinated substrate thereof, i.e. the transcription factor MYC2, explains a molecular mechanism that different species have different levels of sensitivity to Huanglongbing, and provides a breeding strategy where expression levels of the susceptibility gene PUB21 are reduced by knockout (deletion) or modification of the susceptibility gene PUB21 promoter region so as to enhance disease resistance of citrus. The resistance breeding strategy can effectively resist replication and transmission of Huanglongbing pathogens, and has a great potential application value in prevention and control of citrus Huanglongbing.

### BRIEF DESCRIPTION OF THE DRAWINGS

FIG. 1A and FIG. 1B show sequence alignment of PUB21 gene promoters in species with different levels of sensitivity to Huanglongbing. FIG. 1A shows a schematic diagram of Helitron insertions within a PUB21 promoter region of Rutaceae species, where rectangular boxes denote inserted Helitron bodies, and dark-shaded boxes in a Helitron insertion region denote G-box like cis-elements. PUB21 gene promoters of Huanglongbing-susceptible and Huanglongbing-tolerant species (C. sinensis, C. daoxianensis, C. mangshanensis, P. trifoliata, etc.) have Helitron insertions, whereas Huanglongbing-resistant species (M. australasica, Murraya paniculata and Murraya koenigii) have no Helitron insertion. FIG. 1B shows that a CsPUB21 promoter of C. sinensis has a Helitron insertion. Transposon annotations are performed on a genome of C. sinensis using an EDTA method. An annotated transposon library is aligned against a 150-bp insertion sequence within the CsPUB21 promoter using a repeat factor method.

FIGS. 2A-2D show that CsMYC2 may bind to a G-box like motif (CANNTG) within a PUB21 gene promoter region and exerts transcription factor activities to transcriptively activate expression of a PUB21 gene. FIG. 2A shows relative expression levels of a CsPUB21 gene in citrus upon CsMYC2 silencing and transient overexpression of CsMYC2. Values are mean±SEM (n=3) (**P<0.01, Student's t-test). FIG. 2B shows that a CsMYC2 protein directly targets a CsPUB21 gene by binding to a promoter thereof. Chromatin immunoprecipitation (ChIP) reveals relative enrichment multiples of the CsMYC2 protein and DNA regions I, II, III, and IV of a CsPUB21 promoter. Data represent mean±SEM (n=4) (ns denotes no significant difference; *P<0.05, **P<0.01, Student's t-test). FIG. 2C shows electrophoresis migration shift assay (EMSA) of binding of the CsMYC2 protein and a DNA region of the CsPUB21 promoter. FIG. 2D shows DNA-binding activity of the CsMYC2 protein and each G-box like motif in a Helitron transposon. “G” denotes the G-box like motif, and “mG” denotes a mutated G-box like motif.

FIGS. 3A-3G show expression analysis of a CsMYC2 protein transcriptively activating different PUB21 gene promoters. FIG. 3A shows a schematic diagram of CsMYC2 as a construct of an effector and PUB21pro-LUC as a construct of a reporter system. CsPUB21 promoter-luciferase (LUC); CsPUB21^{Δhel} promoter-LUC; MpPUB21 promoter-LUC; and BkPUB21 promoter-LUC serve as constructs of the reporter system. 35S promoter-driven YFP and CsMYC2 are used as constructs of the effector. FIG. 3B shows relative luciferase activity intensity of CsMYC2 activating activities of a PUB21 promoter in tobacco. Data represent mean±SEM (n=8). Lowercase letters indicate significant differences (P<0.05) between different columns based on one-way analysis of variance and Duncan multiple range test. FIGS. 3C and 3D show luciferase imaging of YFP and CsMYC2 for different PUB21 promoter transcription activities in tobacco, where different PUB21 promoter activities are found in a leaf expressing YFP and MYC2. FIGS. 3E and 3F show β-glucuronidase (GUS) staining of YFP and CsMYC2 for different PUB21 promoter transcription activities in tobacco, where different PUB21 promoter activities are found in a leaf expressing YFP and MYC2. FIG. 3G shows relative-glucuronidase activity intensity of CsMYC2 activating different PUB21 promoter activities in tobacco. Values represent mean±SEM (n=8). Lowercase letters indicate significant differences (P<0.05) between different columns based on one-way analysis of variance and Duncan multiple range test.

FIGS. 4A-4D show a partial diagram of deleting Helitron in a PUB21 gene promoter region by Crispr-Cas9 system editing in C. sinensis protoplasts, and analysis of a deleted region, as well as a CsPUB21 gene expression level upon editing, and a relative titer of a pathogen CLas in Huanglongbing-infected protoplasts. FIG. 4A shows PCR/RE-based identification of editing efficiency of a Crispr-Cas9 system in citrus protoplasts (Cas9-CsPUB21^{Δhel} promoter). Band intensities are measured using ImageJ, and editing efficiency is analyzed based on band intensity differences. FIG. 4B shows a partial diagram of CsPUB21 promoter loci and Helitron design in a Crispr-Cas9 construct, and an editing and deletion sequence diagram. Cutting sequences recognized by PAM and Crispr-Cas9 are marked with black underlines respectively, and G-box like motifs in Helitron are marked with black boxes. In protoplasmic editing and transformation, a region where fragments are deleted is marked with black “-”. FIG. 4C shows CsPUB21 gene expression levels upon Helitron editing in citrus protoplasts using a Crispr-Cas9 system. Values represent mean±SEM (n=3) (*P<0.05, Student's t-test). FIG. 4D shows a relative CLas titer upon Helitron editing in protoplasts of Huanglongbing-positive citrus transferred by the Crispr-Cas9 system. Values represent mean±SEM (n=4) (*P<0.05, Student's t-test).

### DETAILED DESCRIPTION

The present disclosure is described in further detail below in connection with specific implementations, and embodiments given are intended only to clarify the present disclosure and not to limit the scope of the present disclosure. The following embodiments are provided as a guide for further improvement by a person of ordinary skill in the art, and do not in any way constitute a limitation of the present disclosure.

Experimental methods in the following embodiments, if not otherwise specified, are conventional methods and are carried out in accordance with the techniques or conditions described in the literature in the art or in accordance with product specifications. The materials, reagents, and the like used in the following embodiments are commercially available, if not otherwise specified.

A pENTR-3C vector in the following embodiments is a product of Invitrogen, and an antibiotic used is a kanamycin antibiotic (Kan). A pH7-YFP-DC vector in the following embodiments is a product of Invitrogen, and an antibiotic used is spectinomycin (Spe).

A pBA-DC-myc vector in the following embodiments is a product of Invitrogen, and an antibiotic used is spectinomycin (Spe). A pH7GWIWG2(II) vector in the following embodiments is a product of Invitrogen, and an antibiotic used is spectinomycin (Spe).

A pGEX-MBP vector in the following embodiments is a product of Invitrogen, and an antibiotic used is Ampicillin (Amp).

A pKGWFS7.0-GUS vector in the following embodiments is a product of Invitrogen, and an antibiotic used is spectinomycin (Spe).

A pGWB435-LUC vector in the following embodiments is a product of Invitrogen, and an antibiotic used is spectinomycin (Spe).

A 35S: YFP control vector in the following embodiments is a vector obtained after cloning a YFP gene into the pBA-DC-myc vector.

A Crispr-Cas9 vector in the following embodiments is recorded in the literature “Yi Zhang, Zhen Liang, Yuan Zong, Yanpeng Wang, Jinxing Liu, Kunling Chen, Jin-Long Qiu, Caixia Gao. Efficient and transgene-free genome editing in wheat through transient expression of CRISPR/Cas9 DNA or RNA. Nat Commun., 2016, 7:12617.”, and an antibiotic used is a kanamycin antibiotic (Kan).

A PE2(V2) vector in the following embodiments is recorded in the literature “Zhen Liang, Yuqing Wu, Yingjie Guo, Sha Wei. Addition of the T5 exonuclease increases the prime editing efficiency in plants. Journal of Genetics and Genomics, 2023, 50(8):582-588.”, and an antibiotic used is a kanamycin antibiotic (Kan).

As for Rutaceae plants used in the following embodiments, Citrus maxima was collected from Danzhou, Hainan Province; Citrus sinensis, Citrus daoxianensis, Microcitrus australiasica, Citrus mangshanensis, Citrus ichangensis, Citrus hongheensis, Citrus medica and Murraya koenigii were all collected from the Citrus Resource Nursery of the Citrus Research Institute of Southwest University, Chongqing; Poncirus polyandra was collected from Kunming, Yunnan Province; and Murraya paniculata was collected from Guangzhou, Guangdong Province. Citrus sinensis, Citrus maxima and Murraya paniculata were grown in a laboratory greenhouse, where the temperature was maintained at 27° C. and the relative humidity was 50%.

Principal reagents in the following embodiments and sources thereof are as follows:

Reagents used for molecular cloning: both EX Taq DNA polymerase and LA Taq DNA polymerase were products from Takara, restriction endonuclease and T4 DNA ligase were products from NEB, homologous recombination ligase Vazyme ClonExpressII One Step Cloning Cl12 was a product from Vazyme, antibiotics used were products from Inolco, 1 Kb DNA marker and 2000 bp DNA marker were both products from Biomed, and SYBR qPCR Mix was a product from TOYOBO.

Reagents used in protein-related experiments: a Cocktail protease inhibitor was a product from Roche, IPTG was a product from Inolco, 40% Acrylamide was a product from Sigma, primary and secondary antibodies were products from Beijing Transgen Biotech, and pre-stained protein molecular weight marker was a product from Easybio.

Kits: Plasmid Mini Prep Kit, Plasmid Maxi Kit, Agarose gel DNA Recovery Kit, and DNA Purification Recycling Kit were products from AxyGen, Plant RNA Mini Prep Kit was a product from Qiagen, ECL luminescent liquid was a product from GE healthcare, and reverse transcription kits were products from Beijing Transgen Biotech. Conventional primers used in the following embodiments were synthesized by Tsingke Biotech, with related sequencing performed; and biotin-labeled EMSA primers were synthesized by Tianyi Huiyuan Biotech.

A nucleotide sequence of a CsPUB21 gene promoter in the following embodiments is as shown in a sequence SEQ ID No. 1, and a nucleotide sequence of Helitron is as shown in SEQ ID No. 4.

A nucleotide sequence of an MpPUB21 gene promoter in the following embodiments is as shown in a sequence SEQ ID No. 5.

A nucleotide sequence of a BkPUB21 gene promoter in the following embodiments is as shown in a sequence SEQ ID No. 6.

An amino acid sequence of a CsMYC2 protein and a coding sequence (CDS) of a CsMYC2 gene in the following embodiments are recorded in the invention patent application document with the publication number CN115160421A.

### Embodiment 1: Detection of Sequences of PUB21 Gene Promoters in Different Rutaceae Species

### I. Sequence Alignment for PUB21 Gene Promoters in Different Rutaceae Species

1. Retrieval of sequences of PUB21 promoters

The National Center for Biotechnology Information (NCBI) of the United States was searched for genomic sequences of plants such as C. sinensis, C. reticulata, C. daoxianensis, C. linwuensis, C. mangshanensis, P. trifoliata, C. maxima, C. hongheensis, C. ichangensis, M. australasica, C. medica, M. paniculata, and B. koenigii. Based on CsPUB21 sequence alignment of C. sinensis, the PUB21 sequence with the highest similarity in other plant genomes was found. Based on position information of the PUB21 sequence in the genome, an upstream 3500-bp promoter sequence was searched forwards, and the found promoter sequence was saved in a text format.
2. Sequence alignment analysis for PUB21 promoters

Multi-sequence alignment analysis was performed on the found nucleotide sequences of the promoters of different Rutaceae species by using DNAMAN sequence analysis software. Through sequence difference analysis, it was discovered that PUB21 promoters of Huanglongbing-susceptible and Huanglongbing-tolerant plants such as C. sinensis, C. reticulata, C. daoxianensis, C. linwuensis, C. mangshanensis, and P. trifoliata had fragment insertions of 87-150 bp at a position 841 bp upstream of a transcriptional start site, while PUB21 promoters of C. maxima, C. hongheensis, C. ichangensis, M. australasica, C. medica, M. paniculata, and B. koenigii highly resistant to Huanglongbing had no insertion of such fragment. By further analyzing sequence characteristics of the inserted fragment, it was found that the inserted fragment was rich in characteristic motifs (G-box like: CANNTG, where N denotes A, T, C, or G) for binding to the transcription factor CsMYC2. This suggested that the inserted fragment could be related to binding to the transcription factor CsMYC2 for further transcriptional activation or inhibition of the expression of the PUB21 gene.

### II. Identification of Inserted DNA Fragment in PUB21 Promoter

Transposon annotation was performed on a C. sinensis genome using an EDTA method. Subsequently, an annotated transposon library was aligned with a 150-bp insertion sequence in the CsPUB21 promoter using a Repeatmaker method. It was found that the insertion sequence belonged to a Class III transposon Helitron. Helitron transposons are repetitive single-stranded (ssDNA) virus-like DNA transposons that significantly impact genomic variation by capturing and mobilizing host genomic sequences.

### Embodiment 2: Regulation of Expression of PUB21 Gene by Binding CsMYC2 to G-Box Like Motif in PUB21 Gene Promoter Region

### I. Construction of Citrus CsMYC2 Gene Silencing and Overexpression Vectors

1. Gene fragment amplification primers are designed, with primer sequences as follows:

CsMYC2-Fw(KpnI):
CAAGGGTACCATGACGGACTACCGGTTACC;
CsMYC2-Rv(XhoI):
CAAGCTCGAGTTATTGGGTATCTCCAACTT;
CsMYC2-800RNAi-Rv(XhoI):
CAAGCTCGAGGGCCAAGTACCAATCTCCAT;

where nucleotide sequences shown underlined were an enzymatic recognition site GGTACC of KpnI and an enzymatic recognition site CTCGAG of XhoI.
2. Using cDNA of C. sinensis as a template, PCR amplification was performed with primers CsMYC2-Fw (KpnI) and CsMYC2-Rv (XhoI) to obtain a gene fragment of 2058 bp.

Using the cDNA of C. sinensis as a template, PCR amplification was performed with primers CsMYC2-Fw (KpnI) and CsMYC2-800RNAi-Rv (XhoI) to obtain a CsMYC2 gene silencing fragment of 800 bp, and a nucleotide sequence thereof was as shown in a sequence SEQ ID No. 7.
3. A pENTR-3C vector was double-digested with restriction endonucleases KpnI and XhoI to obtain a backbone vector. Then, the backbone vector was ligated with the CsMYC2 gene of 2058 bp and the CsMYC2 gene silencing fragment of 800 bp in step 2 respectively using T4 DNA ligase to obtain intermediate vectors pENTR-3C-CsMYC2 and pENTR-3C-CsMYC2-RNAi respectively.
4. The intermediate vector pENTR-3C-CsMYC2 and an expression vector pH7-YFP-DC were ligated through an LR reaction using recombinase (Gateway LR ClonaseII) to obtain an overexpression vector 35S: YFP-CsMYC2.

The intermediate vector pENTR-3C-CsMYC2-RNAi and a gene silencing vector pH7GWIWG2 (II) were ligated through an LR reaction using recombinase (Gateway LR ClonaseII) to obtain a gene silencing vector CsMYC2-RNAi.
5. The overexpression vector 35S: YFP-CsMYC2 and the gene silencing vector CsMYC2-RNAi prepared in step 4 as well as a control vector (35S promoter-driven YFP expression vector 35S: YFP) were electrotransformed into an Agrobacterium GV3101 strain.

### II. CsMYC2 Gene Silencing and Overexpression in Citrus Leaves

1. First, an LB plate without antibiotics was coated with Xanthomonascitrisubsp.citri (Xcc), and cultivated overnight at 28° C. Bacterial cells were collected using sterile water, and an OD₆₀₀ₙₘ value was adjusted to 0.5. Then, an Xcc bacterial solution (which weakened the immunity of citrus, and improved the expression of vector genes in citrus leaves) was injected with a syringe of 1 mL into C. sinensis leaves to obtain C. sinensis leaves injected with the Xcc bacterial solution, and the leaves continued to be placed into a greenhouse for cultivation.
2. The Agrobacterium GV3101 containing the overexpression vector 35S: YFP-CsMYC2, the gene silencing vector CsMYC2-RNAi and the control vector was respectively spread on LB plates containing kanamycin and rifampicin, and the LB plates were cultivated overnight at 28° C. Bacterial cells were collected using an MMA solution (10 mM MgCl2, 10 mM MES, and 150 μM Acetosyringone), and an OD₆₀₀ₙₘ value was adjusted to 1.0. The bacterial cells were left to stand at room temperature for three hours or more.
3. 6 to 8 hours after the Xcc bacterial solution was injected into the leaves, the C. sinensis leaves injected with the Xcc bacterial solution were respectively impregnated with the bacterial solution containing the overexpression vector, the gene silencing vector and the control vector by vacuum injection. Then, the leaves were placed in a plant growth chamber to grow for 72 hours.

### III. CsMYC2 Capable of Effectively Regulating Expression Level of CsPUB21 Gene

1. After 3 days of growth, leaf samples were collected. RNA was extracted from the samples using an RNA extraction kit, and cDNA was synthesized by reverse transcription. Overexpression and silencing genes were detected by fluorescence quantitative PCR, with COX as an internal reference gene. Quantitative primer sequences were as follows:

Citrus CsMYC2 gene detection primer
CsMYC2-qF:
CTACAGCCGACCCCATGAAG;
Citrus CsMYC2 gene detection primer CsMYC2-qR:
TACCCATCGCCCCATCCTAA;
Citrus internal reference gene detection primer
COX-qF:
GTATGCCACGTCGCATTCCAGA;
Citrus internal reference gene detection primer
COX-qR:
GCCAAAACTGCTAAGGGCATTC.

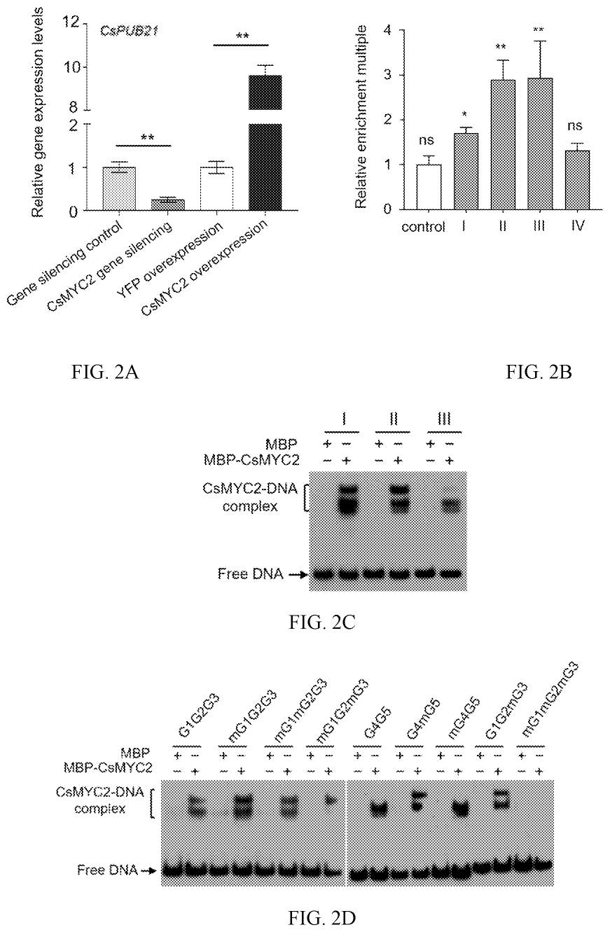

The results are shown in FIG. 2A. The results indicate that the expression level of the CsMYC2 gene in CsMYC2 overexpressed citrus samples was significantly higher than that in the control samples, while the expression level of the CsMYC2 gene in CsMYC2 gene-silenced citrus samples was significantly lower than that in the control samples. This indicates that the constructed overexpression and silencing vectors can effectively overexpress and silence the expression level of the CsMYC2 gene.
2. Meanwhile, based on the cDNA samples from step 1, the expression levels of the CsPUB21 gene in the CsMYC2 overexpressed citrus samples and the CsMYC2 gene-silenced citrus samples were detected by fluorescence quantitative PCR, with COX as an internal reference gene. Quantitative primer sequences were as follows:

Citrus CsPUB21 gene detection primer CsPUB21-qF:
CGTTGGTCGTCGTCTATCGT;
Citrus CsPUB21 gene detection primer CsPUB21-qR:
AATGGAGACTGCGAACTCCG.

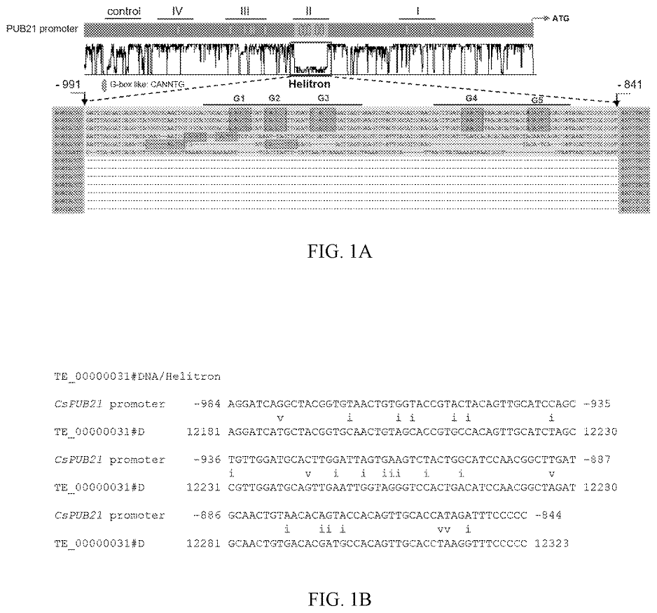

The results are shown in FIG. 1B. The results indicate that the expression level of the CsPUB21 gene in the CsMYC2 gene-silenced citrus samples was significantly lower than that in the control samples, while the expression level of the CsPUB21 gene in the CSMYC2 overexpressed citrus samples was significantly higher than that in the control samples. This indicates that CsMYC2 can effectively regulate the expression level of the CsPUB21 gene.

### IV. Verification of Binding of CsMYC2 to G-Box Like Motif in PUB21 Promoter Region by Using Chromatin Immunoprecipitation (CHIP)

1. Synthesis of detection primers for G-box like motifs of PUB21 promoter

G-box like motifs binding to CsMYC2 in a sequence of a C. sinensis CsPUB21 promoter were predicted using DNAMAN software. A total of 14 motifs were predicted, which were respectively distributed in anterior, middle and posterior regions of the promoter. The motifs were divided into sections I, II, III and IV based on a set of distribution positions of the motifs, and each segment had a length of 150 bp, with an indefinite number of G-box like motifs distributed. 150 bp at the very upstream of the promoter without G-box like motifs was selected as a control region. Quantitative detection primers for the sections I, II, III, IV and the control region were synthesized using the NCBI-prime blast website. Sequences of the quantitative primers were as follows:

| | |
|---|---|
| CsPUB21pro-I-qF: | AGGAGGAAATATAACTCAAA; |
| CsPUB21pro-I-qR: | GAATAAATCAAATCAAAGCT; |
| CsPUB21pro-II-qF: | AGGATCAGGCTACGGTGTAA; |
| CsPUB21pro-II-qR: | CACACAATAAATTACGGGGG; |
| CsPUB21pro-III-qF: | GTGACAGTAAATTCAACTCATC; |
| CsPUB21pro-III-qR: | GCTGAGTTCATTAGTTTCAC; |
| CsPUB21pro-IV-qF: | CAGTCTAATATGAGCGCCTC; |
| CsPUB21pro-IV-qR: | GTGCTTTGAGCGTGATTTAA; |
| CsPUB21pro-ck-qF: | GTCAAGATTAGCATACACACT; |
| CsPUB21pro-ck-qR: | TATCCAATGGATATACGTGT. |

2. Construction of MBP-CsMYC2 protein expression and purification vector, and protein purification
(1) According to the intermediate vector pENTR-3C-CsMYC2 obtained in step I, the intermediate vector pENTR-3C-CsMYC2 was ligated with a protein expression and purification vector pGEX-MBP through an LR reaction using recombinase (Gateway LR ClonaseII) to obtain a CsMYC2 protein expression and purification vector pGEX-CsMYC2-MBP.
(2) The pGEX-CsMYC2-MBP vector constructed in step (1) above was transferred to a BL21 (DE3) competent state for protein induction and purification, and an MBP-CsMYC2 fusion protein was obtained through purification by anti-MBP Tag immunomagnetic beads. Meanwhile, a pGEX-MBP empty vector was transferred into the BL21 (DE3) competent state for protein induction and purification, and an MBP tag protein was obtained through purification by the anti-MBP Tag immunomagnetic beads.
3. Verification of binding enrichment multiple of CsMYC2 and CsPUB21 promoter by using chromatin immunoprecipitation (CHIP)

The binding ability of the MBP-CsMYC2 protein to the sections I, II, III, IV of the PUB21 promoter and the control region was detected in vitro based on a chromatin immunoprecipitation method. For finally obtained DNA products, the concentrations of the sections I, II, III, IV and the control region after enrichment were detected by fluorescence quantitative PCR, with COX as an internal reference gene. See step 1 above for specific primer sequences.

The results are shown in FIG. 2C. The results indicate that compared with a relative enrichment multiple of the control region, the sections I, II and III could significantly bind to and enrich MBP-CsMYC2, where MBP-CsMYC2 had the strongest binding ability with the sections II and III rich in G-box like motifs. This indicates that CsMYC2 can bind to the CsPUB21 promoter by binding to the G-box like motifs.

### V. Verification of Binding of CsMYC2 to G-Box Like Motifs in Helitron by Using Electrophoretic Mobility Shift Assay (EMSA)

1. Synthesis of biotin-labeled EMSA primers of CsPUB21 promoter

Based on the G-box like motif-rich sections I, II and III of the CsPUB21 promoter divided in step IV above, biomarker EMSA detection primers for the sections I, II, III and the control region were designed and synthesized. Primer sequences were as follows:

| | |
|---|---|
| CsPUB21pro-I-Fw: | AGGAGGAAATATAACTCAAA; |
| CsPUB21pro-I-Rv: | GAATAAATCAAATCAAAGCT; |
| CsPUB21pro-II-Fw: | AGGATCAGGCTACGGTGTAA; |
| CsPUB21pro-II-Rv: | CACACAATAAATTACGGGGG; |
| CsPUB21pro-III-Fw: | GTGACAGTAAATTCAACTCATC; |
| CsPUB21pro-III-Rv: | GCTGAGTTCATTAGTTTCAC. |

EMSA detection primers and mutation detection primers were separately designed for the section II rich in G-box like motifs and having Helitron insertions. Primer sequences were as follows:

CsPUB21pro-H123-Fw:
CCGTACTACAGTTGCATCCAGCTGTTGGATGCACTTGGATTAGTG;
CsPUB21pro-H123-Rv:
CACTAATCCAAGTGCATCCAACAGCTGGATGCAACTGTAGTACGG;
CsPUB21pro-Hm123-Fw:
CCGTACTATTGTAACATCCAGCTGTTGGATGCACTTGGATTAGTG;
CsPUB21pro-Hm123-Rv:
CACTAATCCAAGTGCATCCAACAGCTGGATGTTACAATAGTACGG;
CsPUB21pro-Hm1m23-Fw:
CCGTACTATTGTAACATCTTGCAATTGGATGCACTTGGATTAGTG;
CsPUB21pro-Hm1m23-Rv:
CACTAATCCAAGTGCATCCAATTGCAAGATGTTACAATAGTACGG;
CsPUB21pro-Hm12m3-Fw:
CCGTACTATTGTAACATCCAGCTGTTGGATGTTCTAAGATTAGTG;
CsPUB21pro-Hm12m3-Rv:
CACTAATCTTAGAACATCCAACAGCTGGATGTTACAATAGTACGG;
CsPUB21pro-Hm1m2m3-Fw:
CCGTACTATTGTAACATCTTGCAATTGGATGTTCTAAGATTAGTG;
CsPUB21pro-Hm1m2m3-Rv:
CACTAATCTTAGAACATCCAATTGCAAGATGTTACAATAGTACGG;
CsPUB21pro-H12m3-Fw:
CCGTACTACAGTTGCATCTTGCAATTGGATGCACTTGGATTAGTG;
CsPUB21pro-H12m3-Rv:
CACTAATCCAAGTGCATCCAATTGCAAGATGCAACTGTAGTACGG;
CsPUB21pro-Hm45-Fw:
GCTTGATGTTACAATAACACAGTACCACAGTTGCACCAT;
CsPUB21pro-Hm45-Rv:
ATGGTGCAACTGTGGTACTGTGTTATTGTAACATCAAGC;
CsPUB21pro-H4m5-Fw:
GCTTGATGCAACTGTAACACAGTACCATTGTAACACCAT;
CsPUB21pro-H4m5-Rv:
ATGGTGTTACAATGGTACTGTGTTACAGTTGCATCAAGC;
CsPUB21pro-H45-Fw:
GCTTGATGCAACTGTAACACAGTACCACAGTTGCACCAT;
CsPUB21pro-H45-Rv:
ATGGTGCAACTGTGGTACTGTGTTACAGTTGCATCAAGC.

The synthesized primers were mixed into an annealing liquid in a molar ratio of 1:1 to be annealed at 75° C. for 30 minutes, so that a DNA dimer form was formed. Then, the mixture was naturally cooled to room temperature and maintained at −20° C.
2. Verification of binding ability of CsMYC2 to G-box like motifs in Helitron by using electrophoretic mobility shift assay (EMSA)

The binding ability of the MBP-CsMYC2 protein to the sections I, II, and III of the CsPUB21 promoter was detected in vitro based on an electrophoretic mobility shift assay method. The binding ability of MBP-CsMYC2 to the sections I, II, and III and G-box like motifs in Helitron of the section II of the CsPUB21 promoter was determined by electrophoretic mobility. At the same time, an MBP tag protein was used as the control.

The results are shown in FIG. 2C. The results indicate that compared with the control tag protein MBP, which did not form a CsMYC2-DNA complex with the DNA dimer in the sections I, II, and III of the CsPUB21 promoter, the MBP-CsMYC2 protein could significantly bind to the DNA dimers in the sections I, II, and III of the CsPUB21 promoter to form a CsMYC2-DNA complex, resulting in a decrease in the electrophoretic mobility of the DNA dimers and the formation of a distinct CsMYC2-DNA complex binding band.

The binding ability of CsMYC2 to each G-box like motif in Helitron was further analyzed, and the results are shown in FIG. 2D. The results indicate that the MBP-CsMYC2 protein could significantly bind to each G-box like motif in Helitron and form the CsMYC2-DNA complex, however, when completely mutated G-box like motifs were used, this binding ability disappeared and no CsMYC2-DNA complex was formed. This indicates that CsMYC2 can bind to the CsPUB21 promoter by binding to the G-box like motifs. Since inserted Helitron is rich in G-box like motifs, the binding ability of CsMYC2 to the CsPUB21 promoter can be significantly enhanced, and thus the expression of the susceptibility gene CsPUB21 is promoted.

### Embodiment 3: Transcriptional Activation of Expression of PUB21 Gene by CsMYC2 Protein

### I. Construction of PUB21 Promoter Activity Reporter System (LUC/GUS) Vector

1. Promoter fragment amplification primers were designed, with primer sequences as follows:

CsPUB21pro-Fw(BamHI):
CCAATTCAGTCGACTGGATCCTCACGCTCAAAGCACTACGA;
CsPUB21pro-Rv(XhoI):
GCTGGGTCTAGATATCTCGAGGCAAGCCAGCAGTATGCAAG;
MpPUB21pro-Fw(BamHI):
CCAATTCAGTCGACTGGATCCGTGGTCTAATATGAACGCCTC;
MpPUB21pro-Rv(XhoI):
GCTGGGTCTAGATATCTCGAGGCAAGCCAGCAATACGCAAG;
BkPUB21pro-Fw(BamHI):
CCAATTCAGTCGACTGGATCCTCGAATGCCTGGCCGATTTT;
BkPUB21pro-Rv(XhoI):
GCTGGGTCTAGATATCTCGAGCCTTGAACAAAAGAAGCAGGCC;
CsPUB21^{ΔHel}pro-Fw:
CATTTAGATACATGAAATTTATTGTGTGTTTTCAATTAAGG;
CsPUB21^{ΔHel}pro-Rv:
AACACACAATAAATTCATGTATCTAAATGTTATTATCGCG;
C1148pro-Fw:
CTCAGCAACATATTCGGTCAAGAATTTAGGATCGTTATACAAG
C1148pro-Rv:
AGTTGGTTAAACTTGTGGACACGGGGGAAATTTGTGGTCT
Cm87pro-Fw:
CTCAGCAACATATTCGGTCAAGAATTTAGGATCAAACTAC
Cm87pro-Rv:
AGTTGGTTAAACTTGTGGACACGGGAGAAATGCATGTGAT

where nucleotide sequences shown underlined were a homologous arm sequence with a BamHI enzyme digestion recognition site and a homologous arm sequence with an XhoI enzyme digestion recognition site.
2. Using DNA of C. sinensis as a template, PCR amplification was performed with primers CsPUB21pro-Fw (BamHI) and CsPUB21pro-Rv (XhoI) to obtain a CsPUB21pro gene fragment (sequence SEQ ID No. 1) of 2059 bp.

Using DNA of Murraya paniculata as a template, PCR amplification was performed with primers MpPUB21pro-Fw (BamHI) and MpPUB21pro-Rv (XhoI) to obtain an MpPUB21pro gene fragment (sequence SEQ ID No. 5) of 1949 bp.

Using DNA of Murraya koenigii as a template, PCR amplification was performed with primers BkPUB21pro-Fw (BamHI) and BkPUB21pro-Rv (XhoI) to obtain a BkPUB21pro gene fragment (sequence SEQ ID No. 6) of 1758 bp.

Using DNA of C. linwuensis as a template, PCR amplification was performed with primers Cl148pro-Fw and Cl148pro-Rv to obtain a Cl148pro gene fragment (sequence SEQ ID No. 8) of 148 bp.

Using DNA of C. mangshanensis as a template, PCR amplification was performed with primers Cm87pro-Fw and Cm87pro-Rv to obtain a Cm87pro gene fragment (sequence SEQ ID No. 9) of 87 bp.

Using the CsPUB21pro gene fragment as a template, PCR amplification was performed with primers CsPUB21pro-Fw (BamHI) and CsPUB21^{ΔHel}pro-Rv, as well as primers CsPUB21^{ΔHel}pro-Fw and CsPUB21pro-Rv (XhoI), respectively, to obtain gene fragments of 1068 bp and 841 bp respectively; and 100 ng of each of the two fragments was taken for PCR with primers CsPUB21pro-Fw (BamHI) and CsPUB21pro-Rv (XhoI), and then spliced to obtain a Helitron deleted CsPUB21pro gene fragment of 1909 bp, which was designated as a CsPUB21^{ΔHel}pro gene fragment. The CsPUB21^{ΔHel}pro gene fragment was obtained by deleting positions 1069-1218 of a sequence SEQ ID No. 2.

Using the CsPUB21pro gene fragment as a template, PCR amplification was performed with primers CsPUB21pro-Fw (BamHI) and CsPUB21^{ΔHel}pro-Rv, as well as primers CsPUB21^{ΔHel}pro-Fw and CsPUB21pro-Rv (XhoI), respectively, to obtain gene fragments of 1068 bp and 841 bp respectively; and 100 ng of each of a Cl148pro gene fragment and the two fragments of 1068 bp and 841 bp was taken for PCR with primers CsPUB21pro-Fw (BamHI) and CsPUB21pro-Rv (XhoI), and then spliced to obtain a 2057-bp CsPUB21^{ΔHel+cl148}pro gene fragment added to Cl148pro, which was designated as a CsPUB21^{ΔHel+cl148}pro gene fragment (sequence SEQ ID No. 10).

Using the CsPUB21pro gene fragment as a template, PCR amplification was performed with primers CsPUB21pro-Fw (BamHI) and CsPUB21^{ΔHel}pro-Rv, as well as primers CsPUB21^{ΔHel}pro-Fw and CsPUB21pro-Rv (XhoI), respectively, to obtain gene fragments of 1068 bp and 841 bp respectively; and 100 ng of each of a Cm87pro gene fragment and the two fragments of 1068 bp and 841 bp was taken for PCR with primers CsPUB21pro-Fw (BamHI) and CsPUB21pro-Rv (XhoI), and then spliced to obtain a 1996-bp CsPUB21^{ΔHel+cm87}pro gene fragment added to Cm87pro, which was designated as a CsPUB21^{ΔHel+cm87}pro gene fragment (sequence SEQ ID No. 11).
3. A pENTR-3C vector was double-digested with restriction endonucleases BamHI and XhoI to obtain a backbone vector. Then, the backbone vector was ligated with the six fragments from step 2 (the CsPUB21pro gene fragment, the MpPUB21pro gene fragment, the BkPUB21pro gene fragment, the CsPUB21^{ΔHel}pro gene fragment, the CsPUB21^{ΔHel+cl148}pro gene fragment, or the CsPUB21^{ΔHel+cm87}pro gene fragment) by using T4 DNA ligase respectively, to obtain intermediate vectors pENTR-3C-CsPUB21pro, pENTR-3C-MpPUB21pro, pENTR-3C-BkPUB21pro, pENTR-3C-CsPUB21^{ΔHel}pro, pENTR-3C-CsPUB21^{ΔHel+cl148}pro and pENTR-3C-CsPUB21^{ΔHel+cm87}pro, respectively.
4. The intermediate vectors pENTR-3C-CsPUB21pro, pENTR-3C-MpPUB21pro, pENTR-3C-BkPUB21pro, pENTR-3C-CsPUB21^{ΔHel}pro, pENTR-3C-CsPUB21^{ΔHel+cl148}pro and pENTR-3C-CsPUB21^{ΔHel+cm87}pro were ligated with an expression vector pKGWFS7.0-GUS through an LR reaction using recombinase (Gateway LR ClonaseII) to obtain recombinant vectors pKGWFS7.0-CsPUB21pro-GUS, pKGWFS7.0-MpPUB21pro-GUS, pKGWFS7.0-BkPUB21pro-GUS, pKGWFS7.0-CsPUB21^{ΔHel}pro-GUS, pKGWFS7.0-CsPUB21^{ΔHel+cl148}pro-GUS and pKGWFS7.0-CsPUB21^{ΔHel+cm87}pro-GUS, respectively.

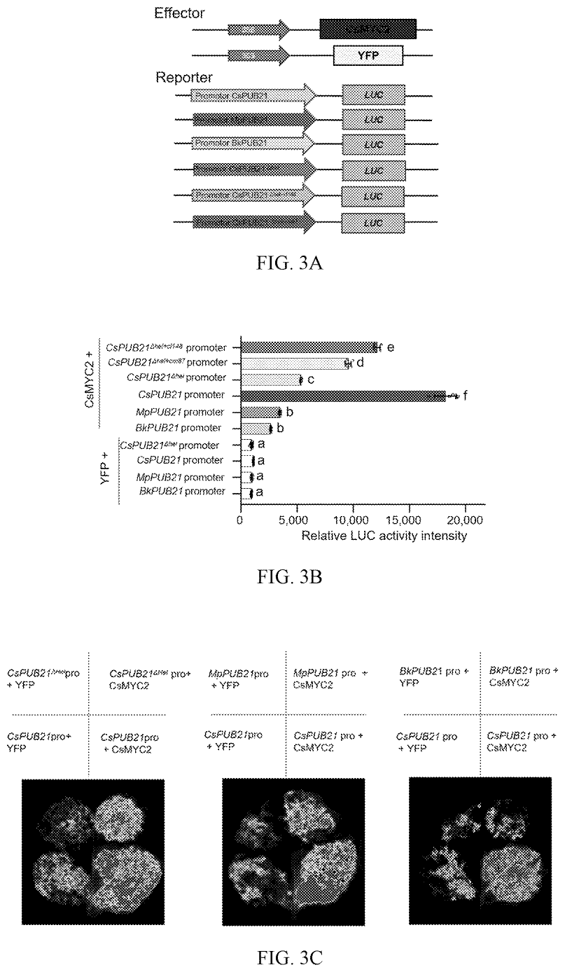

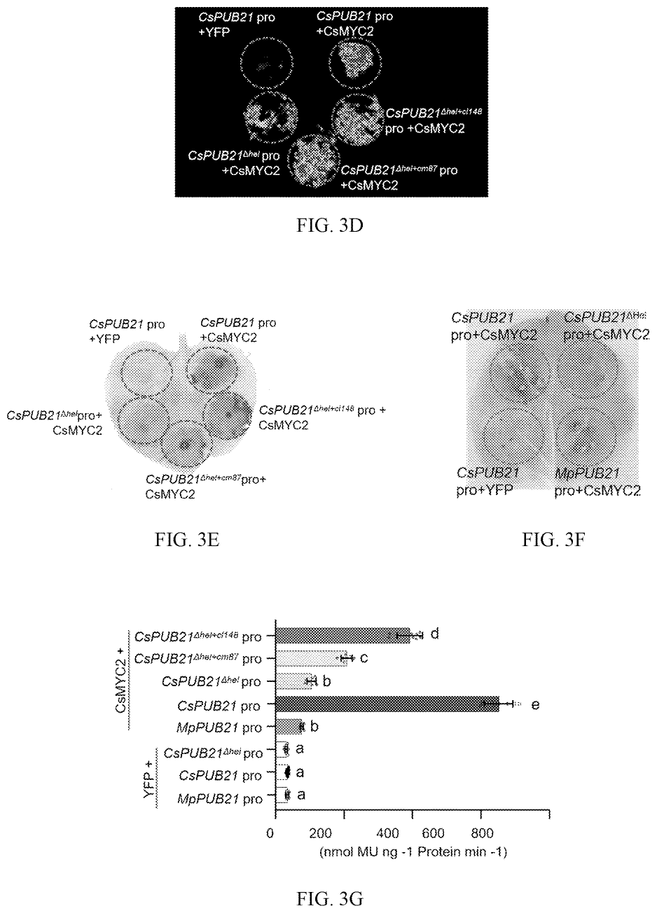

The intermediate vectors pENTR-3C-CsPUB21pro, pENTR-3C-MpPUB21pro, pENTR-3C-BkPUB21pro, pENTR-3C-CsPUB21^{ΔHel}pro, pKGWFS7.0-CsPUB21^{ΔHel+cl148}pro-GUS and pKGWFS7.0-CsPUB21^{ΔHel+cm87}pro-GUS were ligated with an expression vector pGWB435-LUC through an LR reaction using recombinase (Gateway LR ClonaseII) to obtain recombinant vectors pGWB435-CsPUB21pro-LUC, pGWB435-MpPUB21pro-LUC, pGWB435-BkPUB21pro-LUC, pGWB435-CsPUB21^{ΔHel}pro-LUC, pGWB435-CsPUB21^{ΔHel+cl148}pro-LUC and pGWB435-CsPUB21^{ΔHel+cm87}pro-LUC, respectively. FIG. 3A shows schematic diagrams of each recombinant vector.
5. The recombinant vectors prepared in step 4 were electrotransformed into the Agrobacterium GV3101 strain.

### II. Verification of Reporter System for CsMYC2 Transcriptional Activation of PUB21-LUC in Tobacco Leaves

1. Agrobacterium GV3101 having the overexpression vector 35S: YFP-CsMYC2 or the control vector 35S: YFP and Agrobacterium GV3101 having LUC reporter system vectors pGWB435-CsPUB21pro-LUC, pGWB435-MpPUB21pro-LUC, pGWB435-BkPUB21pro-LUC, pGWB435-CsPUB21^{ΔHel}pro-LUC, pGWB435-CsPUB21^{ΔHel+cl148}pro-LUC and pGWB435-CsPUB21^{ΔHel+cm87}pro-LUC were spread on LB plates having spectinomycin and rifampicin, and cultivated overnight at 28° C. Bacterial cells were collected using an MMA solution. An OD₆₀₀ₙₘ value was adjusted to 1.0, and the bacterial cells were allowed to stand at room temperature for three hours or more.
2. Bacterial solutions were mixed evenly at a volume ratio of 1:1 based on the combinations shown in FIG. 3C. The mixed bacterial solution was injected into tobacco leaves using a syringe of 1 mL, and the tobacco leaves continued to be placed in a greenhouse for cultivation for 48 h.
3. The impact of CsMYC2-YFP on transcriptional activities of different PUB21 promoters was observed using a low-temperature cryogenic fluorescence imager.

Results are shown in FIG. 3B, FIG. 3C, and FIG. 3D. FIG. 3B shows that CsMYC2-YFP could activate the PUB21 promoter to exhibit luciferase activity compared to a control YFP, with CsMYC2-YFP exhibiting the strongest activation for the CsPUB21 promoter and the most significant luciferase activity intensity. The degree of Helitron full-length or fragment deletion was directly correlated with relative LUC activity intensity. Compared to the CsPUB21 promoter, the activity intensity of CsPUB21^{ΔHel}pro luciferase with Helitron full-length deletion was reduced by 72% or more, while the activity intensities of cis-element exchange mutants CsPUB21^{ΔHel+cl148}pro luciferase and CsPUB21^{ΔHel+cm87}pro luciferase produced by inserting two MYC2 cis-elements of intermediate citrus species, i.e. 148 bp of C. linwuensis (cl148) or 87 bp of C. mangshanensis (cm87) into CsPUB21^{Δhel} were reduced by 30% or more and 45% or more, respectively. For the MpPUB21 and BkPUB21 promoters, which naturally lack Helitron insertions, the activity intensity of luciferase thereof was approximately 16% to 20% that of the luciferase of the CsPUB21 promoter.

FIG. 3C and FIG. 3D visually show luciferase imaging of YFP and CSMYC2 for transcription activities of different PUB21 promoters in tobacco. CsMYC2-YFP exhibited the strongest activation for the CsPUB21 promoter and the most significant luciferase activity intensity, while the activity intensity of the Helitron-deleted CsPUB21^{ΔHel}pro luciferase was significantly weakened. Meanwhile, the activity intensities of luciferase of the MpPUB21 and BkPUB21 promoters lacking Helitron insertions were lower. These results indicate that the transcription activation of the expression of the PUB21 gene by the CsMYC2 transcription factor mainly functions through Helitron inserted in the promoter region.

### III. Verification of Reporter System for CsMYC2 Transcriptional Activation of PUB21-GUS in Tobacco Leaves

1. Agrobacterium GV3101 having the overexpression vector 35S: YFP-CsMYC2 or the control vector 35S: YFP and Agrobacterium GV3101 having GUS reporter system vectors pKGWFS7.0-CsPUB21pro-GUS, pKGWFS7.0-MpPUB21pro-GUS, pKGWFS7.0-BkPUB21pro-GUS, pKGWFS7.0-CsPUB21^{ΔHel}pro-GUS, pKGWFS7.0-CsPUB21^{ΔHel+cl148}pro-GUS and pKGWFS7.0-CsPUB21^{ΔHel+cm87}pro-GUS were spread on LB plates having spectinomycin and rifampicin, and cultivated overnight at 28° C. Bacterial cells were collected using an MMA solution. An OD 600 nm value was adjusted to 1.0, and the bacterial cells were allowed to stand at room temperature for three hours or more.
2. Bacterial solutions were mixed evenly at a volume ratio of 1:1 based on a combination shown in FIG. 3F. The mixed bacterial solution was injected into tobacco leaves using a syringe of 1 mL, and the tobacco leaves continued to be placed in a greenhouse for cultivation for 48 h.
3. The impact of CsMYC2-YFP on transcriptional activities of different PUB21 promoters was observed by β-glucuronidase (GUS) staining.

Results are shown in FIG. 3E, FIG. 3F, and FIG. 3G. FIG. 3E and FIG. 3F show that the same leaf was employed. Compared to the control YFP, CsMYC2-YFP could significantly activate the PUB21 promoter and exhibit a significant GUS staining response, where CsMYC2-YFP exhibited the strongest activation for the CsPUB21 promoter and the most significant relative GUS activity intensity. The activity intensity of CsPUB21^{ΔHel+cl148}pro and CsPUB21^{ΔHel+cm87}pro luciferase was reduced, while the activity intensity of the Helitron-deleted CsPUB21^{ΔHel}pro luciferase was significantly weakened. Meanwhile, the relative GUS activity intensity of the MpPUB21 promoter lacking Helitron insertions was lower. This confirms that the inserted Helitron transposon plays a primary role in transcriptional activation of the expression of PUB21 by the CsMYC2 transcription factor.

FIG. 3G further shows different degrees of relative GUS activity intensities exhibited by different PUB21 promoters activated by CSMYC2-YFP, where the degree of Helitron full-length or fragment deletion was directly correlated with the relative GUS activity intensity. Compared to the CsPUB21 promoter, the activity intensity of CsPUB21^{ΔHel+cl148}pro luciferase of a cis-element exchange mutant was reduced by about 35%, the activity intensity of CsPUB21^{ΔHel+cm87}pro luciferase was reduced by 67% or more, while the activity intensity of CsPUB21^{ΔHel}pro luciferase with Helitron full-length deletion was reduced by 84% or more, which was almost comparable to the activity intensity of luciferase of the MpPUB21 promoter lacking Helitron insertions.

### Embodiment 4: Knockout of CsPUB21 Promoter Fragment by Crispr-Cas9 Editor to Effectively Resist CLas

To further confirm the impact of the number of G-box like motifs within the PUB21 promoter on a pathogenic mechanism of a Huanglongbing susceptibility gene PUB21, the present disclosure deleted and edited, based on a Crispr-Cas9 editing system, a G-box like motif of a Helitron insertion fragment within the CsPUB21 promoter from susceptible citrus protoplasts, so as to observe the disease resistance against the citrus Huanglongbing pathogen. The specific steps were as follows:

### I. Optimization of Promoter in Crispr-Cas9 Editing System

Due to the fact that promoters of initial sgRNA and Cas9 in an original Crispr-Cas9 editing system were derived from OsU3 and MaUbi in rice and corn, the editing efficiency in citrus was low. Therefore, the present disclosure optimized promoters in the Crispr-Cas9 editing system.
1. Promoter amplification primers were designed, with primer sequences as follows:

CsU6-Cas9-Fw:
AAAACGACGGCCAGTGCCAAGCTTGCGCTCAGGAGCCGGTTGAA;
CsU6-Cas9-Rv:
GCTATTTCTAGCTCTAAAACCGAGACCTTGTGTTGGTCTCG;
CmYLCV-Cas9-
Fw: TACTGCTTGCTGCTAAGCTTTGGCAGACATACTGTCCCAC;
CmYLCV-Cas9-Rv:
GATCCGTCGACAAGCTCCTAGGAAGCTTAGCTCTTACCTGTTTTCG;

where the nucleotide sequences shown underlined were homologous arm sequences at both ends of an original promoter of the Crispr-Cas9 editing system. Using the DNA of Citrus sinensis as a template, PCR amplification was performed with primers CsU6-Cas9-Fw and CsU6-Cas9-Rv to obtain a 665-bp CsU6 promoter gene fragment with a homologous arm. The nucleotide sequence thereof was as shown in SEQ ID No. 2. Based on the CmYLCV promoter sequence downloaded from NCBI, a 465-bp CmYLCV promoter gene fragment with a homologous arm was commercially synthesized at Tsingke Biotech. The nucleotide sequence thereof was as shown in SEQ ID No. 3.
2. An amplification primer of an intermediate fragment was designed, with primer sequences as follows:

Gap-sgRNA-Cas9-Fw:
CGAGACCAACACAAGGTCTCGGTTTTAGAGCTAGAAATAGC;
Gap-sgRNA-Cas9-Fw:
GTGGGACAGTATGTCTGCCAAAGCTTAGCAGCAAGCAGTA;

where the nucleotide sequences shown underlined were homologous arm sequences at both ends of an intermediate fragment in the Crispr-Cas9 editing system.

Using a prime editing vector PE2 (V2) as a template, PCR amplification was performed with primers Gap-sgRNA-Cas9-Fw and Gap-sgRNA-Cas9-Fw to obtain a 414-bp Gap-sgRNA intermediate fragment sequence having a homologous arm. The nucleotide sequence thereof was as shown in SEQ ID No. 12.
3. A Crispr-Cas9 vector was single-digested using restriction endonuclease HindIII to obtain a backbone vector fragment of 16,657 bp and a fragment of 2033 bp. The backbone vector fragment of 16,657 bp was recovered. The recovered backbone vector fragment of 16,657 bp was single-digested again using restriction endonuclease AvrII to obtain a backbone vector fragment of 14,696 bp and a fragment of 2000 bp. The backbone vector fragment of 14,696 bp was recovered.
4. 100 ng of each of the CsU6 promoter, Gap-sgRNA, and CmYLCV promoter fragments obtained from PCR in steps 1 and 2 was taken for PCR with primers CsU6-Cas9-Fw and CmYLCV-Cas9-Rv, and spliced to obtain a fusion gene fragment of 1550 bp. The nucleotide sequence thereof was as shown in SEQ ID No. 13.
5. Finally, the fusion fragment of 1652 bp in step 4 was recombined with the backbone vector of 14,696 bp obtained in step 3 using a Cl12 homologous recombinase. Upon ligation and transformation identification, a promoter-optimized editing vector PHUE-CsU6-Gap-CmYLCV-Cas9 was obtained.

### II. Construction of Cas9-PUB21Pro Editing Vector

1. A Cas9-CsPUB21pro-sgRNA amplification primer was designed, with primer sequences as follows:

Cas9-CsPUB21pro-sgRNA-Fw:
GTTGAGCTGGATGCAACTGTAGTA;
Cas9-CsPUB21pro-sgRNA-Rv:
AAACTACTACAGTTGCATCCAGCT;

where the nucleotide sequences shown underlined were sticky ends at both ends of an original Gap region of the optimized editing vector PHUE-CsU6-Gap-CmYLCV-Cas9.

Primers Cas9-CsPUB21pro-sgRNA-Fw and Cas9-CsPUB21pro-sgRNA-Rv were mixed at a volume ratio of 1:1, treated at 95° C. for 5 min, and then cooled naturally to room temperature to form double-stranded DNA with sticky ends.
2. The PHUE-CsU6-Gap-CmYLCV-Cas9 vector was single-digested with restriction endonuclease BsaI to obtain a linear backbone vector. Then, the linear backbone vector was ligated with the double-stranded DNA fragment with the sticky ends obtained in step 1 using the Cl12 homologous recombinase to obtain the Crispr-Cas9 editing vector PHUE-CsU6-CsPUB21pro-CmYLCV-Cas9.

The Crispr-Cas9 editing vector PHUE-CsU6-CsPUB21pro-CmYLCV-Cas9 contained an sgRNA gene expression cassette and a Cas9 gene expression cassette, expressing sgRNA and Cas9 protein targeting the Helitron region within the CsPUB21 promoter. In the sgRNA gene expression cassette, the target sequence of sgRNA was positions 1105-1124 of the sequence SEQ ID No. 1, and the CsU6 promoter was used as the promoter used to initiate the sgRNA gene expression. The CmYLCV promoter was used as the promoter used to initiate the Cas9 gene expression in the Cas9 gene expression cassette.

III. Knockout of Fragment Containing G-Box Like Motif in CsPUB21 Promoter by Crispr-Cas9 editing can reduce CLas titer in protoplasts of diseased leaves
1. Large-scale extraction of recombinant plasmid of PHUE-CsU6-CsPUB21pro-CmYLCV-Cas9

PHUE-CsU6-CsPUB21pro-CmYLCV-Cas9 was transferred into a DH5a competent state, and streaked overnight on a solid LB plate containing a Kan antibiotic. Single colonies were picked and shaken overnight in a 10 mL liquid LB containing a Kan antibiotic. A bacterial solution was transferred to a 200 mL liquid LB containing a Kan antibiotic and shaken for 12 hours. Bacterial cells were collected, and a Tiangen (endotoxin-free) plasmid large-scale extraction kit was used for plasmid extraction.
2. The large-scale extracted plasmids in step 1 were transformed into citrus protoplasts. First, diseased young citrus (sweet orange) leaves (diseased citrus trees were grown in a laboratory greenhouse, and the disease status was determined by detecting the amount of the pathogen CLas before the experiment) were collected, and veins were removed. The leaves were cut into thin strips, soaked in citrus leaf enzymatic hydrolysate, and shaken in the dark for 5 hours. The enzymatic hydrolysate was filtered with a gauze. After two times of washing, protoplast precipitates were collected and placed on ice for about 30 minutes. The transformation of PHUE-CsU6-CsPUB21pro-CmYLCV-Cas9 recombinant plasmids was performed in a 37° C. water bath for 30 minutes using PEG4000-mediated protoplast transformation. Upon the transformation, a supernatant was removed by brief centrifugation. A protoplast culture medium was added, and cultivation was performed in the dark for 48 hours. Meanwhile, the transformed PHUE-CsU6-Gap-CmYLCV-Cas9 vector was used as the control.
3. After 48 hours of protoplast cultivation, samples were collected. Protoplast DNA was extracted using a CTAB method to synthesize and edit site-enriched specific detection primers. Primer sequences were as follows:

CsPUB21pro-Id-Fw: GTAAAAAAATGCACGGCC;
CsPUB21pro-Id-Rv: TTACTATCATTTCTTCTAACC.

Using the extracted protoplast DNA as a template, PCR amplification was performed with editing identification primers CsPUB21pro-Id-Fw and CsPUB21pro-Id-Rv to obtain a CsPUB21pro-Id gene fragment of 491 bp. PCR products were treated with SfcI restriction endonucleases carried in the CsPUB21pro-sgRNA sequence. The editing efficiency of the PCR products after enzymatic digestion was detected by agarose gel electrophoresis. Wild-type genomic amplification products without editing were used as controls. If editing occurred, bands of the same size as the amplified fragments could be detected.

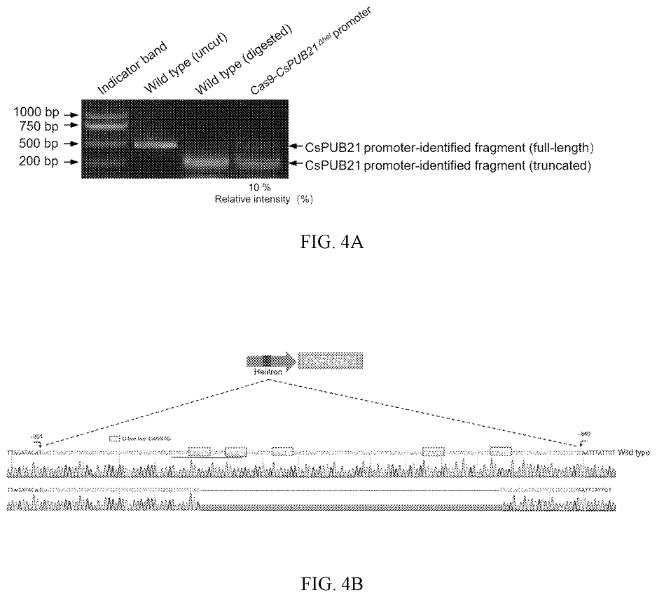

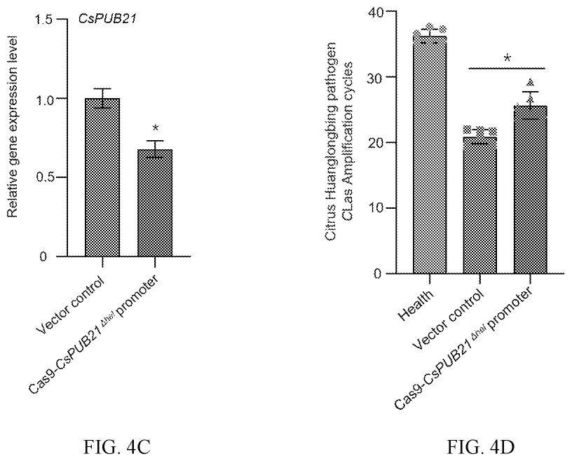

The results are shown in FIG. 4A. The results indicate that compared with the uncut and digested wild-type CsPUB21pro-Id fragments, the digested edited group CsPUB21pro-Id had a band of 491 bp, which was the same as the amplified fragment. The band intensity was measured using ImageJ, and editing efficiency of 10% was shown based on the measured band intensity.

Meanwhile, the amplified CsPUB21pro-Id gene fragment of 491 bp was ligated to TA clones for single-colony sequencing.

The results are shown in FIG. 4B. The results indicate that in citrus protoplasts, an 8 nt position downstream of an sgRNA-PAM recognition site in the Helitron region of the CsPUB21 promoter could be effectively cut using the Crispr-Cas9 editing system, thereby successfully deleting a fragment of 84 bp. The deleted fragment was located at positions 1113-1196 of the sequence SEQ ID No. 1, and the editing efficiency thereof was 10%.

The expression level of the CsPUB21 gene in citrus protoplasts after Cas9-CsPUB21pro editing was detected by fluorescence quantitative PCR. The specific steps referred to step III in Embodiment 2.

The results are shown in FIG. 4C. The results indicate that compared with a control transformed empty vector group, the expression level of the CsPUB21 gene was significantly reduced after Cas9-CsPUB21pro editing. It indicates that the successful knockout of the Helitron region of the CsPUB21 promoter by Crispr-Cas9 editing can significantly reduce the expression level of the susceptibility gene CsPUB21.
4. Using the protoplast DNA extracted in step 3 as a template, the concentration of the pathogen CLas in the citrus protoplasts after Cas9-CsPUB21pro editing was detected by fluorescence quantitative PCR. With COX as an internal reference gene, quantitative primer sequences were as follows:

Specific detection primer HLBas for Huanglongbing
pathogen CLas:
GTCGAGCGCGTATGCAATACG;
Specific detection primer HLBr for Huanglongbing
pathogen CLas:
GCGTTATCCCGTAGAAAAAGGTAG;
Citrus internal reference gene detection primer
COXf:
GGTATGCCACGTCGCATTCCAGA;
Citrus internal reference gene detection primer
COXr:
GCCAAAACTGCTAAGGGCATTC.

The results are shown in FIG. 4D. The results indicate that the CLas concentration in the diseased protoplasts was significantly reduced after Cas9-CsPUB21pro editing, and had a significant difference from the CLas concentration in the control transformed empty vector group. This indicates that after knockout of the Helitron region of the CsPUB21 promoter by Crispr-Cas9 editing, the resistance of citrus to the Huanglongbing pathogen could be effectively enhanced, and an effective anti-CLas effect was exhibited.

The present disclosure is described in detail above. For those skilled in the art, the present disclosure can be practiced in a wide range of equivalent parameters, concentrations and conditions without departing from the purpose and scope of the present disclosure and without unnecessary experimentation. Although particular embodiments are given in the present disclosure, it should be understood that further improvements can be made to the present disclosure. In summary, in accordance with the principles of the present disclosure, the present application is intended to encompass any changes, uses or improvements to the present disclosure, including changes out of the scope of what has been disclosed in the present application and made with conventional techniques known in the art. The application of some of the essential features may be carried out according to the scope of the following appended claims.

### INDUSTRIAL APPLICABILITY

The present disclosure identifies a citrus or Nicotiana genus gene fragment capable of regulating Huanglongbing resistance of plants, and a mechanism thereof for directly interacting with Huanglongbing susceptibility gene PUB21 promoter-PUB21-MYC2 to enhance disease resistance, and provides a genetic resource effectively against Huanglongbing pathogen replication and transmission. In practical applications, by editing the plant susceptibility gene PUB21 promoter region fragment, a binding region to an MYC2 transcriptional gene can be reduced, and the expression of the PUB21 susceptibility gene can be reduced and/or eliminated. Therefore, the resistance of recipient plants to Huanglongbing and a transmission carrier thereof, Asian Diaphorina citri Kuwayama, is enhanced to cultivate Huanglongbing-resistant plant varieties. The present disclosure is of great significance to the prevention and control of Huanglongbing in plants.

1. A DNA molecule, wherein the DNA molecule is any one of a1) to a3):
a1) a DNA molecule with a sequence as shown in SEQ ID No. 1;
a2) a DNA molecule having a same function as the DNA molecule defined in a1), obtained by replacement and/or insertion and/or deletion of one or several bases; and
a3) a DNA molecule that is at least 70%, at least 75%, at least 80%, at least 85%, at least 90%, at least 95%, at least 96%, at least 97%, at least 98% or at least 99% identical to and has a same function as the DNA molecule defined in a1).

2. The DNA molecule according to claim 1, wherein replacement and/or insertion and/or deletion of the one or several bases refer(s) to replacement and/or insertion and/or deletion of no more than 10 bases.

3. A biomaterial, wherein the biomaterial is a substance obtained by reducing or eliminating an expression and/or function of a full-length or partial fragment of the DNA molecule according to claim 1, wherein the full-length or partial fragment of the DNA molecule comprises a characteristic motif that binds to a transcription factor MYC2.

4. The biomaterial according to claim 3, wherein reducing or eliminating the expression and/or function of the full-length or partial fragment of the DNA molecule refers to treating the full-length or partial fragment of the DNA molecule with gene knockout technology, gene silencing technology, inactivation mutation technology, PROTAC technology or small molecule inhibitors.

5. A method for preparing the biomaterial according to claim 3, comprising treating a characteristic motif in a full-length or partial fragment of the DNA molecule that binds to a transcription factor MYC2 with gene knockout technology, gene silencing technology, inactivation mutation technology, PROTAC technology or small molecule inhibitors, wherein the DNA molecule is any one of a1) to a3), wherein the gene knockout technology comprises zinc finger nucleases (ZFN) technology, transcription activator-like effector (TALE) technology, CRISPR/Cas technology or TALE-CRISPR/Cas technology, wherein the inactivation mutation technology comprises mutating the characteristic motif in the full-length or partial fragment of the DNA molecule that binds to the transcription factor MYC2, so as to prevent the transcription factor MYC2 from binding to the characteristic motif, wherein a mutation form may be deletion mutation and/or insertion mutation and/or base substitution, wherein the DNA molecule is any one of a1) to a3).

6. The method according to claim 5, wherein the gene knockout technology uses a CRISPR/Cas9 gene editing vector, and the CRISPR/Cas9 gene editing vector expresses an sgRNA and Cas9 protein targeting the full-length or partial fragment of the DNA molecule, wherein the DNA molecule is any one of a1) to a3).

7. The method according to claim 6, wherein a target sequence of the sgRNA is as shown in positions 1105-1124 of a sequence SEQ ID NO: 1.

8. A method according to the biomaterial of claim 3 is applied in any one of A1) to A6):
A1) enhancing Huanglongbing resistance of plants, plant parts, or plant cells;
A2) preparation of products for enhancing Huanglongbing resistance of plants, plant parts, or plant cells;
A3) cultivation of Huanglongbing-resistant plants, plant parts, or plant cells;
A4) preparation of products for cultivating Huanglongbing-resistant plants, plant parts, or plant cells;
A5) prevention and control of Huanglongbing in plants, plant parts, or plant cells; and
A6) preparation of products for preventing and controlling Huanglongbing in plants, plant parts, or plant cells.

9. A gene regulation system, configured to prepare the biomaterial according to claim 3.

10. The gene regulation system according to claim 9, wherein the gene regulation system treats a characteristic motif in a full-length or partial fragment of the DNA molecule that binds to a transcription factor MYC2 with gene knockout technology, gene silencing technology, inactivation mutation technology, PROTAC technology, or small molecule inhibitors, wherein the DNA molecule is any one of a1) to a3).

11. The gene regulation system according to claim 10, wherein the gene regulation system comprises a nucleic acid molecule and an enzyme protein, wherein the nucleic acid molecule is an sgRNA molecule, and the enzyme protein is a Cas protein or an ortholog of Cas, wherein the enzyme protein is selected from Cas3, Cas9, Cas12a, Cas12b, Cas13a, Cas13b, Cas13c, Cas13e, Cas13f, CasX, or IscB proteins or orthologs thereof.

12. The gene regulation system according to claim 11, wherein a vector used in the gene regulation system is a CRISPR/Cas9 gene editing vector that expresses an sgRNA and Cas9 protein targeting the full-length or partial fragment of the DNA molecule, wherein the DNA molecule is any one of a1) to a3).

13. A method of the gene regulation system according to claim 9 is applied in any one of A1) to A6):
A1) enhancing Huanglongbing resistance of plants, plant parts, or plant cells;
A2) preparation of products for enhancing Huanglongbing resistance of plants, plant parts, or plant cells;
A3) cultivation of Huanglongbing-resistant plants, plant parts, or plant cells;
A4) preparation of products for cultivating Huanglongbing-resistant plants, plant parts, or plant cells;
A5) prevention and control of Huanglongbing in plants, plant parts, or plant cells; and
A6) preparation of products for preventing and controlling Huanglongbing in plants, plant parts, or plant cells.

14. A kit comprising the gene regulation system according to claim 9.

15. A method of the kit according to claim 14 is applied in any one of A1) to A6):
A1) enhancing Huanglongbing resistance of plants, plant parts, or plant cells;
A2) preparation of products for enhancing Huanglongbing resistance of plants, plant parts, or plant cells;
A3) cultivation of Huanglongbing-resistant plants, plant parts, or plant cells;
A4) preparation of products for cultivating Huanglongbing-resistant plants, plant parts, or plant cells;
A5) prevention and control of Huanglongbing in plants, plant parts, or plant cells; and
A6) preparation of products for preventing and controlling Huanglongbing in plants, plant parts, or plant cells.

16. A PUB21 promoter, comprising the DNA molecule according to claim 1.

17. A method for enhancing Huanglongbing resistance of plants, plant parts, or plant cells or for cultivating Huanglongbing-resistant plants, plant parts, or plant cells, comprising administering to recipient plants, plant parts, or plant cells the gene regulation system according to claim 9 or the kit comprising the gene regulation system according to claim 9, such that an expression and/or function of a full-length or partial fragment of the DNA molecule in a PUB21 gene promoter in the recipient plants, plant parts, or plant cells are reduced or eliminated, wherein the DNA molecule is any one of a1) to a3).

18. A method for enhancing Huanglongbing resistance of plants, plant parts, or plant cells or for cultivating Huanglongbing-resistant plants, plant parts, or plant cells, comprising the step of administering the biomaterial according to claim 3 to recipient plants, plant parts, or plant cells.

19. The method according to claim 17, wherein the plants are any one of P1) to P8):
P1) monocotyledonous or dicotyledonous plants;
P2) the dicotyledonous plants being plants of the order Rutales or Solanales;
P3) the plants of the order Rutales being plants of the Rutaceae family;
P4) the plants of the order Solanales being plants of the Solanaceae family;
P5) the plants of the Rutaceae family being Citrus genus plants;
P6) the plants of the Solanaceae family being Nicotiana genus plants;
P7) the Citrus genus plants being citrus; and
P8) the Nicotiana genus plants being tobacco.

20. A Huanglongbing-resistant plant, plant part, or plant cell prepared by the method according to claim 17.


SEQUENCE LISTING (13 sequences)

SEQ ID NO: 1 - DNA, 2059 bp, unidentified
  source 1..2059: mol_type = other DNA
gtcaagattagcatacacactaactattctaagtttgtattaatttcctctgcgaattaacattccctgtatgcatatatgccctttccttaaattctttatcgtatgtatatccatgattatataaacttgcacacgtatatccattggatatacatacatacatacatacatacatacatacatacatacatacatatatatgtatgtatgtatgtatgtatgtatgtatgtgtatgtatttgtatatatgaaacaaatttgtgaagtgtgaattacgttcgattgcctggccaattttagatgctttgcagcgtctgtttattatttacatatattggtatctagaattttttttcaacaccttattttccaaatcttaacagtctaatatgagcgcctcaattagaaattaatgggttttttttttttttaatctcttgtgtttcttccatttcaactggccctacatattttctttcaaattcatgttaaaagattcttctatatagattttaaatcacgctcaaagcactacgattgttttaattaacattgatctattctgtaataattaatctgtttaacgaattaaaatagttgtattctaggatataagtcagaaactattgttcattgaaattattgattctatgattaaattacaacctgtggacaaaaatatgatctcagggattgtcattttgtatatgcatctcgatcaattcatctagaaagaaaataaatgggatgtgtcagagtgaaatgaattggcttaattagtccaataaatttgtgacagtaaattcaactcatctgatgtttatgtattaaataagtaaaaaaatgcacggccttgctttcttttctaacccacagtcggaacgcgttgaacagttggattttacccagagtccagttgatcatttgcattaacagacggggtgcgcgtttgggccgcgtaataagaatatacaaatgtgaaactaatgaactcagcaacatattcggtcaagtaaaactgaaaaaaagaaaaaaaagaaacgcgataataacatttagatacatgatttaggatcaggctacggtgtaactgtggtaccgtactacagttgcatccagctgttggatgcacttggattagtgaagtctactggcatccaacggcttgatgcaactgtaacacagtaccacagttgcaccatagatttcccccgtaatttattgtgtgttttcaattaaggatatagtttattaatttgtattaagtaagtccacaagtttaaccaacttgactaaaataggtttggttagaagaaatgatagtaaagtagagcaatggaaaggaaagagaaaaagaagaaatgagtgaaaatgaacatcaatcttatttttgttatttgattggacaaaaaatttatctttctcttgttatttttttcctctctttttttttttttttgtttcttcataccaaacttggggctatttggtagattggaaagagaaagggaataaaattaataaaggaggaaatataactcaaatcacatggctgactaactgtgctgttaccattattcgaaagagtaagccaggccaagtcagccagaccgccgactcgtacgttgacgagctatttttctttcgttccaactgtgtcaaagctttgatttgatttattctaaaaataataaaagcacacacatatatacccaacacttcgtcttaccgtttgaaacattcaacggtcaaacattcaaatccctcgttacacaacattgatctaaaacatcttcgtattatgttcaaatctgaagattgtatttattattattttttttccaagttttgaatgttgaaccgaccgcgtgtcgcttgtatataaaaccgatcaattctccttccattccttccttaacacatacaacacaacaaaacttaaaacttccttcacatcaaaattttcaaaaagcgcaattaacaattagctgcttgcatactgctggcttgcttattttgttcaaggatatattatagttgtacgcaatatataaca

SEQ ID NO: 2 - DNA, 665 bp, unidentified
  source 1..665: mol_type = other DNA
gcgctcaggagccggttgaatttgattgttgtttgatgtttaggtatgcttacaattttttactaaataaggtaataattgttccttttattttattcatttagcttggtacttgtcgtaatctatctccgtcaatgtcagcttcttctgtgtcggaagaagctgaaaaatgttgttgaaggccctttcatttattattatcattattatgtttaattttgttcggcaaatgtgtactaatttggttcgaatcgttcgatctcataatacaaacatgtaatgagttatttatgacgtcatggatgcggttagtttgcgtggtctgaaatttcaaccaaattattcaatcatggtggtggccggctggtgcctatcccttgattgaaatatgcaattaaaattctcgtaataatgttgatttgttctatatacttgaattgatggtattaattattgttataaaagcactaacttgtttgagaaaggggataactaaaaggtaataataataataataaaataaagaaaaggcccaacacatgggcgccctatctgttgggccaaatgccataagaggatccagctagcccgttgaagaaaatcccacatcgaaagaaaaaactgaataaacatggtctatatatacaaggactcccaggttggtt

SEQ ID NO: 3 - DNA, 465 bp, unidentified
  source 1..465: mol_type = other DNA
tggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagctt

SEQ ID NO: 4 - DNA, 1949 bp, unidentified
  source 1..1949: mol_type = other DNA
gtcaagaacggcacacgcactaattattcaatctaaagttgtatttcctcaacgagttaacactccctgtatacatatatggcttagaaaaacaattggctccacttaatttctttatcgtatgtatatccatgattatatatcaactagtacacgtatattcatcggaatgaatgaataactgaatttttttttttaaaagacatgatgtaataaataactagagttggtactttttacaatttctgttccgttaattgtaatctaaattcgaattacattcgattgcctggccgatttgagatgctttgcaacgtctgtttattatttatatatcttactttctagaatttttttcttcctcctgaaaagtgaaaaaaaaaaaaacactttctagagttttcaacaccttatttttcaaatcttagtggtctaatatgaacgcctcaatcagaaattaatgagtttatttatttatttatttgaatctcatgtgtttcttccatttcatgctggccctacatattttctttcgaattcatggcagaagattcttcgacatagatgttaaatcacgctaaaagcactacgattattttaattaacattgatcttttctataatagttaatctgtttaatgaattaaaatacttgtattctatgatgtaactcagaaactattgttcactgaaattatttattctataattaaattacaacccgtctagacaaatatgatctcagggattgtcattttgtatgtgcatttcaatcaattcatctgaaagcaaaaatatgggatgtgccacaatgaaatgatttggcttaattagtccaataaaatttttgatagtaaattcaattcatctaatgtttatgtattaaataaataaaaaatggacggccttgctttcttttccaacccacagcacgcgtttaacagttagatattacccagaatcatttgcactaacagttgggtgcgcgtgtgggccgcgtaataggattgtccaaatgtgaaacaatgaactcagcaactcactcagtcaagtaaactgaaaggaaaaaaaaaaagtggaaaagggagaataacatgtagatacctaatttcttatgtgttttcaattaaggatacagtttattaatttgtattaagtaggttcacaagtttaaccaacctgactaaaataccctttcttttccacgtttggctggaagaaataataagagaagaaagtcgaagaagagaaaatgagtaaaaaaatagaatcgatcttatttttattttttgtttggacaaaaaatctatctttctctccttaattcattcttattttcccctctcctcttttctttttatatttcttcaaaccaaacatgggttcatttggtagattcgaaagagaaaaggaagaaattgataaaggagaaaaacgtaactcaagccacatggctgaccgtgctgttatcatcgttcgaaagagtaagctaagccaagtcagccagaccgccgacccgtacgttgaccagccatttttctttcttacaaactgtgtcaaagctttgatttgatttattctaaaataataaaagcacacaccgatataccaacagtcatcttaccgtttcaaacattcaacggtcaaacattcaaaatccctcgctacacaacattgatctaaaatatcgtggtattaaattcaaatctgaagattgtatttttatatattttttttcttccaagttttgaatgttgaaccgaccgcgtttctctctgtatataaaacccaccagttctccttctattccttccttaagacacacaacaaaaattataatttcctttacatcaaaattcaaaaagcacaagtaataattagcttcttgcgtattgctggcttgcttcttttgttcaaggatatattatagttgtactgaatatatcaca

SEQ ID NO: 5 - DNA, 150 bp, unidentified
  source 1..150: mol_type = other DNA
gatttaggatcaggctacggtgtaactgtggtaccgtactacagttgcatccagctgttggatgcacttggattagtgaagtctactggcatccaacggcttgatgcaactgtaacacagtaccacagttgcaccatagatttcccccgt

SEQ ID NO: 6 - DNA, 1758 bp, unidentified
  source 1..1758: mol_type = other DNA
gtcaagattagcatacacactaattattcaatctaaatttgtatttcctctgcaaattaacattccctgtatacatatattgctgacaaaaacaattggccctactttttttgtttatcgtatgtatatccatgattgtatatcaacttgtacacgtgtatccatctgaataagtgaataattgatattttttttaaaacatgatgtaataaataacaagagtatgtacttttgacaatttctgttccgttaattgtaaataaaatcgtgaaatatgaataacattcgaatgcctggccgattttagatttatttgcagtgcgtgtttattatttatatatacttctttctagaattttcaacactctattttccaaatcttaacagtgtaatatgaacgcctcaatcagaaatttatgagtttatgtttttttttttaatctcctgtgttccttccatttcagctggccctacatatttgctttcaaactcatgctaaaagattcttcgatgtagatattaaattatgctgaaagcactacgattattttaattaatattgatctcttctataatggttaaattttttaatcaattaaaatataactcaaaactatttttcattgaaattatttattctacaattaaattacaacctgcatagaaaaatatgatctcaaagattgtcattttatacatgcatttcaatcaattcatctaaaaacacaaaaaaaagggatgtgtcacaatgaaatgatttggcttaattagtacaatataattgtgatagtaaattcaattcatctaatgtttatgtattaaataaataaagaatgcatgcgctgctttcttttctaactcaaattctgtaatacagtacgcgtttagcagtcagaaatcacccagaacccatttaatcatttgtgctgacagatgggggagcgcgtttgggccgcgtaagaagattatacaaatgtttaacaatgaactcagcatgagcaactccgtcaagtaaactctaaaaaaacataggagcaggataataacatctaggtacctagtttcttatttattttcaattagggatacaggttcttaacttgtaataagtaagtccacgagtttaaccaacctgagtacctgactggtaaaataaaaaaataatgggggccgtttagtagatttgaaagagaaatgggaagaaattgataaaggaggaaaacatagctcaaagccacacggctgactgtgcagttatcaatattcgaaagagtaagataagctaagtcagccagtccgccgaccccgtacgttgacgagctattttttccttcttcgtaactgtgtcaaaggtttgatttattctaaaataattaaagcacacacctacatacctaacagttcattttaccggttcaagcattcaacggtcaaagattcaaatccttcgttacacaacattgatcttaaatatctttgtattatattcaaatctgaaagttgtatattttttctttttttcttttttttttccaagttttgaatgttgaaccgaccgcgtttcccttgtatataaaacccaccaatgctccttctattccctccttaagacacacaacaaaaacaaaaactaaaactaaaaataaaacctccattacatcaaaattcaaaaagcacaagcaataattaacttcttgcatattgctggcctgcttcttttgttcaaggatatattatagctgtactgagtatataaca

SEQ ID NO: 7 - DNA, 800 bp, unidentified
  source 1..800: mol_type = other DNA
atgacggactaccggttaccttcaacgatgaatctatggactgacgataacggctctgtaatggaagctttcatgagctctgatttgaccggtatttggccaccttcgcagtcttctgcatctacagccgaccccatgaagactcacatctcttcttcatcccaacaacagcaacaacaacaacaattcttcaatcaagaaacactccagcaacgccttcagcaattgattgaaggctctcgtgagggctggacctacgccatattctggcaatcctcgtgcgattactccggctcctctatgttaggatggggcgatgggtattacaaaggagaaggagagaaaggaaaatcatcaaaaattaaaacgtcgtcagctgcggagcaggagcatcgtaagaaggtactgcgcgaacttaactcgttgatttctggatcaacgtcttcgccgactgatgatgctgttgatgaagaggttacagatacagaatggttctttttgatttcgatgactcagtctttttacgtgaccggaggcggtggcggtggcgggctgccgggtcaagcgtattttggtaacagtccggtttgggttagcggagcggagcgactggcgaattcaggttgtgaccgggcaagacaggggcaggttttcgggttacagactttggtttgtataccgtcggcaaatggggttgttgaattggggtccactgaggtgattattcaaaactcggatctgatgaataaggttcgatttttgtttaattttaatggcagtatggagattggtacttggcc

SEQ ID NO: 8 - DNA, 148 bp, unidentified
  source 1..148: mol_type = other DNA
aatttaggatcgttatacaagaatcgcagatgcgtcacttgtctgccaaattagttggatgcgacttggattagtgggtcccatttacatccatcggttggatctgatcacacacaagatctacaatcagaccacaaatttcccccgt

SEQ ID NO: 9 - DNA, 87 bp, unidentified
  source 1..87: mol_type = other DNA
aatttaggatcaaactacaattgtgatccagttggacgacttagtggagtcattggcgttcaactacatcacatgcatttctcccgt

SEQ ID NO: 10 - DNA, 2057 bp, unidentified
  source 1..2057: mol_type = other DNA
gtcaagattagcatacacactaactattctaagtttgtattaatttcctctgcgaattaacattccctgtatgcatatatgccctttccttaaattctttatcgtatgtatatccatgattatataaacttgcacacgtatatccattggatatacatacatacatacatacatacatacatacatacatacatacatatatatgtatgtatgtatgtatgtatgtatgtatgtgtatgtatttgtatatatgaaacaaatttgtgaagtgtgaattacgttcgattgcctggccaattttagatgctttgcagcgtctgtttattatttacatatattggtatctagaattttttttcaacaccttattttccaaatcttaacagtctaatatgagcgcctcaattagaaattaatgggttttttttttttttaatctcttgtgtttcttccatttcaactggccctacatattttctttcaaattcatgttaaaagattcttctatatagattttaaatcacgctcaaagcactacgattgttttaattaacattgatctattctgtaataattaatctgtttaacgaattaaaatagttgtattctaggatataagtcagaaactattgttcattgaaattattgattctatgattaaattacaacctgtggacaaaaatatgatctcagggattgtcattttgtatatgcatctcgatcaattcatctagaaagaaaataaatgggatgtgtcagagtgaaatgaattggcttaattagtccaataaatttgtgacagtaaattcaactcatctgatgtttatgtattaaataagtaaaaaaatgcacggccttgctttcttttctaacccacagtcggaacgcgttgaacagttggattttacccagagtccagttgatcatttgcattaacagacggggtgcgcgtttgggccgcgtaataagaatatacaaatgtgaaactaatgaactcagcaacatattcggtcaagtaaaactgaaaaaaagaaaaaaaagaaacgcgataataacatttagatacataatttaggatcgttatacaagaatcgcagatgcgtcacttgtctgccaaattagttggatgcgacttggattagtgggtcccatttacatccatcggttggatctgatcacacacaagatctacaatcagaccacaaatttcccccgtaatttattgtgtgttttcaattaaggatatagtttattaatttgtattaagtaagtccacaagtttaaccaacttgactaaaataggtttggttagaagaaatgatagtaaagtagagcaatggaaaggaaagagaaaaagaagaaatgagtgaaaatgaacatcaatcttatttttgttatttgattggacaaaaaatttatctttctcttgttatttttttcctctctttttttttttttttgtttcttcataccaaacttggggctatttggtagattggaaagagaaagggaataaaattaataaaggaggaaatataactcaaatcacatggctgactaactgtgctgttaccattattcgaaagagtaagccaggccaagtcagccagaccgccgactcgtacgttgacgagctatttttctttcgttccaactgtgtcaaagctttgatttgatttattctaaaaataataaaagcacacacatatatacccaacacttcgtcttaccgtttgaaacattcaacggtcaaacattcaaatccctcgttacacaacattgatctaaaacatcttcgtattatgttcaaatctgaagattgtatttattattattttttttccaagttttgaatgttgaaccgaccgcgtgtcgcttgtatataaaaccgatcaattctccttccattccttccttaacacatacaacacaacaaaacttaaaacttccttcacatcaaaattttcaaaaagcgcaattaacaattagctgcttgcatactgctggcttgcttattttgttcaaggatatattatagttgtacgcaatatataaca

SEQ ID NO: 11 - DNA, 1996 bp, unidentified
  source 1..1996: mol_type = other DNA
gtcaagattagcatacacactaactattctaagtttgtattaatttcctctgcgaattaacattccctgtatgcatatatgccctttccttaaattctttatcgtatgtatatccatgattatataaacttgcacacgtatatccattggatatacatacatacatacatacatacatacatacatacatacatacatatatatgtatgtatgtatgtatgtatgtatgtatgtgtatgtatttgtatatatgaaacaaatttgtgaagtgtgaattacgttcgattgcctggccaattttagatgctttgcagcgtctgtttattatttacatatattggtatctagaattttttttcaacaccttattttccaaatcttaacagtctaatatgagcgcctcaattagaaattaatgggttttttttttttttaatctcttgtgtttcttccatttcaactggccctacatattttctttcaaattcatgttaaaagattcttctatatagattttaaatcacgctcaaagcactacgattgttttaattaacattgatctattctgtaataattaatctgtttaacgaattaaaatagttgtattctaggatataagtcagaaactattgttcattgaaattattgattctatgattaaattacaacctgtggacaaaaatatgatctcagggattgtcattttgtatatgcatctcgatcaattcatctagaaagaaaataaatgggatgtgtcagagtgaaatgaattggcttaattagtccaataaatttgtgacagtaaattcaactcatctgatgtttatgtattaaataagtaaaaaaatgcacggccttgctttcttttctaacccacagtcggaacgcgttgaacagttggattttacccagagtccagttgatcatttgcattaacagacggggtgcgcgtttgggccgcgtaataagaatatacaaatgtgaaactaatgaactcagcaacatattcggtcaagtaaaactgaaaaaaagaaaaaaaagaaacgcgataataacatttagatacataatttaggatcaaactacaattgtgatccagttggacgacttagtggagtcattggcgttcaactacatcacatgcatttctcccgtaatttattgtgtgttttcaattaaggatatagtttattaatttgtattaagtaagtccacaagtttaaccaacttgactaaaataggtttggttagaagaaatgatagtaaagtagagcaatggaaaggaaagagaaaaagaagaaatgagtgaaaatgaacatcaatcttatttttgttatttgattggacaaaaaatttatctttctcttgttatttttttcctctctttttttttttttttgtttcttcataccaaacttggggctatttggtagattggaaagagaaagggaataaaattaataaaggaggaaatataactcaaatcacatggctgactaactgtgctgttaccattattcgaaagagtaagccaggccaagtcagccagaccgccgactcgtacgttgacgagctatttttctttcgttccaactgtgtcaaagctttgatttgatttattctaaaaataataaaagcacacacatatatacccaacacttcgtcttaccgtttgaaacattcaacggtcaaacattcaaatccctcgttacacaacattgatctaaaacatcttcgtattatgttcaaatctgaagattgtatttattattattttttttccaagttttgaatgttgaaccgaccgcgtgtcgcttgtatataaaaccgatcaattctccttccattccttccttaacacatacaacacaacaaaacttaaaacttccttcacatcaaaattttcaaaaagcgcaattaacaattagctgcttgcatactgctggcttgcttattttgttcaaggatatattatagttgtacgcaatatataaca

SEQ ID NO: 12 - DNA, 414 bp, unidentified
  source 1..414: mol_type = other DNA
cgagaccaacacaaggtctcggttttagagctagaaatagcaagttaaaataaggctagtccgttatcaacttgaaaaagtggcaccgagtcggtgcttttttttttcgttttgcattgagttttctccgtcgcatgtttgcagttttattttccgttttgcattgaaatttctccgtctcatgtttgcagcgtgttcaaaaagtacgcagctgtatttcacttatttacggcgccacattttcatgccgtttgtgccaactatcccgagctagtgaatacagcttggcttcacacaacactggtgacccgctgacctgctcgtacctcgtaccgtcgtacggcacagcatttggaattaaagggtgtgatcgatactgcttgctgctaagctttggcagacatactgtcccac

SEQ ID NO: 13 - DNA, 1550 bp, unidentified
  source 1..1550: mol_type = other DNA
aaaacgacggccagtgccaagcttgcgctcaggagccggttgaatttgattgttgtttgatgtttaggtatgcttacaattttttactaaataaggtaataattgttccttttattttattcatttagcttggtacttgtcgtaatctatctccgtcaatgtcagcttcttctgtgtcggaagaagctgaaaaatgttgttgaaggccctttcatttattattatcattattatgtttaattttgttcggcaaatgtgtactaatttggttcgaatcgttcgatctcataatacaaacatgtaatgagttatttatgacgtcatggatgcggttagtttgcgtggtctgaaatttcaaccaaattattcaatcatggtggtggccggctggtgcctatcccttgattgaaatatgcaattaaaattctcgtaataatgttgatttgttctatatacttgaattgatggtattaattattgttataaaagcactaacttgtttgagaaaggggataactaaaaggtaataataataataataaaataaagaaaaggcccaacacatgggcgccctatctgttgggccaaatgccataagaggatccagctagcccgttgaagaaaatcccacatcgaaagaaaaaactgaataaacatggtctatatatacaaggactcccaggttggttgcgagaccaacacaaggtctcggttttagagctagaaatagcaagttaaaataaggctagtccgttatcaacttgaaaaagtggcaccgagtcggtgcttttttttttcgttttgcattgagttttctccgtcgcatgtttgcagttttattttccgttttgcattgaaatttctccgtctcatgtttgcagcgtgttcaaaaagtacgcagctgtatttcacttatttacggcgccacattttcatgccgtttgtgccaactatcccgagctagtgaatacagcttggcttcacacaacactggtgacccgctgacctgctcgtacctcgtaccgtcgtacggcacagcatttggaattaaagggtgtgatcgatactgcttgctgctaagctttggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttc